(19) United States
(12) Patent Application Publication
(10) Pub. No.: US 2007/0197626 A1
(43) Pub. Date: Aug. 23, 2007

(54) Indoline derivatives

(75) Inventors: Elke Dittrich-Wengenroth, Wuppertal (DE); Stephan Siegel, Wuppertal (DE); Michael Woltering, Hilden (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 10/568,887

(22) PCT Filed: Aug. 16, 2004
(86) PCT No.: PCT/EP04/09175

§ 371 Date: Aug. 4, 2006

(30) Foreign Application Priority Data
Aug. 18, 2003 (DE) 10337839.1

(51) Int. Cl. A61K 31/404; C07D 209/04

(52) U.S. Cl. 514/415; 548/509

(57) ABSTRACT

The present invention relates to novel indoline derivatives, to processes for their preparation and to their use in medicaments, in particular as potent PPAR-delta-activating compounds for the prophylaxis and/or treatment of cardiovascular disorders, in particular of dyslipidaemias, arteriosclerosis and coronary heart diseases.

The present application relates to novel indoline derivatives, to processes for their preparation and to their use in medicaments, in particular as potent PPAR-delta-activating compounds for the prophylaxis and/or treatment of cardiovascular disorders, in particular of dyslipidaemias, arteriosclerosis and coronary heart diseases.

In spite of many successful therapies, coronary heart diseases (CHDs) remain a serious public health problem. Treatment with statins, which inhibit HMG-CoA reductase, very successfully lowers the LDL cholesterol plasma concentration, resulting in a significant reduction of the mortality of patients at risk; however, convincing treatment strategies for the therapy of patients having an unfavourable HDL/LDL cholesterol ratio and/or hyper-triglyeridaemia are still not available to date.

Currently, fibrates are the only therapy option for patients of these risk groups. They act as weak agonists of the peroxisome-proliferator-activated receptor (PPAR)-alpha (Nature 1990, 347, 645-50). A disadvantage of fibrates which have hitherto been approved is that their interaction with the receptor is only weak, requiring high daily doses and causing considerable side-effects.

For the peroxisome-proliferator-activated receptor (PPAR)-delta (Mol. Endocrinol. 1992, 6, 1634-41), first pharmacological findings in animal models indicate that potent PPAR-delta-agonists may likewise lead to an improvement in the HDL/LDL cholesterol ratio and in hypertriglyceridaemia.

It was an object of the present invention to provide novel compounds suitable for use as PPAR-delta modulators.

Indoline derivatives as phospholipase inhibitors for the treatment of inflammable disorders are claimed in WO 99/43672, WO 99/43654 and WO 99/43651.

The present invention provides compounds of the general formula (I) 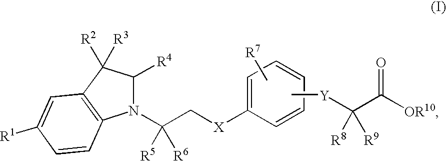
in which
R¹ represents phenyl or represents 5- or 6-membered heteroaryl having up to two heteroatoms from the group consisting of N, O and/or S, which radicals may for their part each be substituted by one to three identical or different substitutents selected from the group consisting of halogen, cyano, nitro, (C₁-C₆)-alkyl (which for its part may be substituted by hydroxyl), (C₁-C₆)-alkoxy, trifluoromethyl, trifluoromethoxy, (C₁-C₆)-alkylsulphonyl, (C₁-C₆)-alkanoyl, (C₁-C₆)-alkoxycarbonyl, carboxyl, amino, (C₁-C₆)-acylamino, mono- and di-(C₁-C₆)-alkylamino,
R² and R³ are identical or different and independently of one another represent hydrogen or (C₁-C₄)-alkyl or together with the carbon atom to which they are attached form a 3- to 7-membered spiro-linked cycloalkyl ring,
R⁴ represents hydrogen or (C₁-C₄)-alkyl,
R⁵ and R⁶ represent hydrogen or together with the carbon atom to which they are attached form a carbonyl group,
R⁷ represents hydrogen, (C₁-C₄)-alkyl, (C₁-C₄)-alkoxy or halogen,
R⁸ and R⁹ are identical or different and independently of one another represent hydrogen or (C₁-C₄)-alkyl,
R¹⁰ represents hydrogen or represents a hydrolyzable group which may be degraded to the corresponding carboxylic acid,
X represents O, S or N—R¹¹
and
Y represents a bond
or
X represents a bond
and
Y represents O, S or N—R¹¹, where
   R¹¹ represents in each case hydrogen, (C₁-C₄)-alkyl or (C₁-C₄)-alkanoyl,
      and their salts, solvates and solvates of the salts.

In the context of the invention, in the definition of R¹⁰, a hydrolyzable group means a group which, in particular in the body, causes the —C(O)OR¹⁰ grouping to be converted into the corresponding carboxylic acid (R¹⁰=hydrogen). Such groups are, by way of example and by way of preference: benzyl, (C₁-C₆)-alkyl or (C₃-C₈)-cycloalkyl which are in each case optionally mono- or polysubstituted by identical or different substitutents from the group consisting of halogen, hydroxyl, amino, (C₁-C₆)-alkoxy, carboxyl, (C₁-C₆)-alkoxycarbonyl, (C₁-C₆)-alkoxycarbonylamino or (C₁-C₆)-alkanoyloxy, or in particular (C₁-C₄)-alkyl which is optionally mono- or polysubstituted by identical or different substitutents from the group consisting of halogen, hydroxyl, amino, (C₁-C₄)-alkoxy, carboxyl, (C₁-C₄)-alkoxycarbonyl, (C₁-C₄)-alkoxycarbonylamino or (C₁-C₄)-alkanoyloxy.

In the context of the invention, (C₁-C₆)-alkyl and (C₁-C₄)-alkyl represent a straight-chain or branched alkyl radical having 1 to 6 and 1 to 4 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl and tert-butyl.

In the context of the invention, (C₃-C₈)-cycloalkyl represents a monocyclic cycloalkyl group having 3 to 8 carbon atoms. The following radicals may be mentioned by way of example and by way of preference: cyclopropyl, cyclobutyl, cyclopentyl and cyclohexyl.

In the context of the invention, (C₁-C₆)-alkoxy and (C₁-C₄)alkoxy represent a straight-chain or branched alkoxy radical having 1 to 6 and 1 to 4 carbon atoms, respectively. Preference is given to a straight-chain or branched alkoxy radical having 1 to 4 carbon atoms. The following radicals may be mentioned by way of example and by way of preference: methoxy, ethoxy, n-propoxy, isopropoxy and tert-butoxy.

In the context of the invention, (C₁-C₆)-alkoxycarbonyl and (C₁-C₄)-alkoxycarbonyl represent a straight-chain or branched alkoxy radical having 1 to 6 and 1 to 4 carbon atoms, respectively, which radical is attached via a carbonyl group. Preference is given to a straight-chain or branched alkoxycarbonyl radical having 1 to 4 carbon atoms. The following radicals may be mentioned by way of example and by way of preference: methoxycarbonyl, ethoxycarbonyl, n-propoxycarbonyl, isopropoxycarbonyl and tert-butoxycarbonyl.

In the context of the invention, (C₁-C₆)-alkoxycarbonylamino and (C₁-C₄)-alkoxycarbonylamino represent an amino group having a straight-chain or branched alkoxycarbonyl substitutent which has 1 to 6 and 1 to 4 carbon atoms, respectively, in the alkoxy radical and which is attached via the carbonyl group. Preference is given to an alkoxycarbonylamino radical having 1 to 4 carbon atoms. The following radicals may be mentioned by way of example and by way of preference: methoxycarbonylamino, ethoxycarbonylamino, n-propoxycarbonylamino and tert-butoxycarbonylamino.

In the context of the invention, (C₁-C₆)-alkanoyl and (C₁-C₆)-alkanoyl represent a straight-chain or branched alkyl radical having 1 to 6 and 1 to 4 carbon atoms, respectively, which carries a doubly attached oxygen atom in the 1-position and is attached via the 1-position. Preference is given to a straight-chain or branched alkanoyl radical having 1 to 4 carbon atoms. The following radicals may be mentioned by way of example and by way of preference: formyl, acetyl, propionyl, n-butyryl, i-butyryl, pivaloyl and n-hexanoyl.

In the context of the invention, (C₁-C₆)-alkanoyloxy and (C₁-C₄)-alkanoyloxy represent a straight-chain or branched alkyl radical having 1 to 6 and 1 to 4 carbon atoms, respectively, which carries a doubly attached oxygen atom in the 1-position and is attached in the 1-position via a further oxygen atom. Preference is given to an alkanoyloxy radical having 1 to 4 carbon atoms. The following radicals may be mentioned by way of example and by way of preference: acetoxy, propionoxy, n-butyroxy, i-butyroxy, pivaloyloxy, n-hexanoyloxy.

In the context of the invention, mono-(C₁-C₆)-alkylamino and mono-(C₁-C₄)-alkylamino represent an amino group having a straight-chain or branched alkyl substitutent of 1 to 6 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino and tert-butylamino.

In the context of the invention, di-(C₁-C₆)-alkylamino and di-(C₁-C₄)-alkylamino represent an amino group having two identical or different straight-chain or branched alkyl substitutents having in each case 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

In the context of the invention, (C₁-C₆)-acylamino represents an amino group having a straight-chain or branched alkanoyl substitutent which has 1 to 6 carbon atoms and is attached via the carbonyl group. Preference is given to an acylamino radical having 1 or 2 carbon atoms. The following radicals may be mentioned by way of example and by way of preference: formamido, acetamido, propionamido, n-butyramido and pivaloylamido.

In the context of the invention, (C₁-C₆)-alkylsulphonyl represents a straight-chain or branched alkylsulphonyl radical having 1 to 6 carbon atoms. Preference is given to a straight-chain or branched alkylsulphonyl radical having 1 to 4 carbon atoms. The following radicals may be mentioned by way of example and by way of preference: methylsulphonyl, ethylsulphonyl, n-propylsulphonyl, isopropylsulphonyl, tert-butylsulphonyl, n-pentylsulphonyl and n-hexylsulphonyl.

In the context of the invention, 5- or 6-membered heteroaryl having up to 2 identical or different heteroatoms from the group consisting of N, O and S represents a monocyclic aromatic heterocycle (heteroaromatic) which is attached via a ring carbon atom or, if appropriate, via a ring nitrogen atom of the heteroaromatic. The following radicals may be mentioned by way of example: furyl, pyrrolyl, thienyl, pyrazolyl, imidazolyl, thiazolyl, oxazolyl, isoxazolyl, isothiazolyl, pyridyl, pyrimidinyl, pyridazinyl and pyrazinyl. 5- or 6-membered heteroaryl radicals having up to two nitrogen atoms are preferred, such as imidazolyl, pyridyl, pyrimidinyl, pyridazinyl and pyrazinyl.

In the context of the invention, halogen includes fluorine, chlorine, bromine and iodine. Preference is given to chlorine or fluorine.

Depending on the substitution pattern, the compounds according to the invention can exist in stereoisomeric forms which are either like image and mirror image (enantiomers) or not like image and mirror image (diastereomers). The invention relates both to the enantiomers or diastereomers and to their respective mixtures. The racemic forms, like the diastereomers, can be separated in a known manner into the stereoisomerically uniform components.

Furthermore, certain compounds can be present in tautomeric forms. This is known to the person skilled in the art, and such compounds are likewise included in the scope of the invention.

The compounds according to the invention can also be present as salts. In the context of the invention, preference is given to physiologically acceptable salts.

Physiologically acceptable salts can be salts of the compounds according to the invention with inorganic or organic acids. Preference is given to salts with inorganic acids such as, for example, hydrochloric acid, hydrobromic acid, phosphoric acid or sulphuric acid, or to salts with organic carboxylic or sulphonic acids such as, for example, acetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenedisulphonic acid.

Physiologically acceptable salts can also be salts of the compounds according to the invention with bases, such as, for example, metal or ammonium salts. Preferred examples are alkali metal salts (for example sodium salts or potassium salts), alkaline earth metal salts (for example magnesium salts or calcium salts), and also ammonium salts which are derived from ammonia or organic amines, such as, for example, ethylamine, di- or triethylamine, ethyldiisopropylamine, monoethanolamine, di- or triethanolamine, dicyclohexylamine, dimethylaminoethanol, dibenzylamine, N-methylmorpholine, dihydroabietylamine, 1-ephenamine, methylpiperidine, arginine, lysine, ethylenediamine or 2-phenylethylamine.

The compounds according to the invention can also be present in the form of their solvates, in particular in the form of their hydrates.

Preference is given to compounds of the general formula (I) in which
R¹ represents phenyl which may be mono- or disubstituted by identical or different substitutents selected from the group consisting of halogen, cyano, nitro, (C₁-C₄)alkyl (which for its part may be substituted by hydroxyl), (C₁-C₄)-alkoxy, trifluoromethyl, trifluoromethoxy, (C₁-C₄)-alkanoyl, amino, mono- and di-(C₁-C₄)-alkylamino,
R² and R³ are identical or different and represent (C₁-C₄)-alkyl or together with the carbon atom to which they are attached form a 4- to 6-membered spiro-linked cycloalkyl ring,
R⁴ represents hydrogen,
R⁵ and R⁶ represent hydrogen or together with the carbon atom to which they are attached form a carbonyl group,
R⁷ represents hydrogen, (C₁-C₄)-alkyl, (C₁-C₄)-alkoxy, fluorine or chlorine,
R⁸ and R⁹ independently of one another represent hydrogen or methyl,
R¹⁰ represents hydrogen,
X represents O or S
and
Y represents a bond
or
X represents a bond
and
Y represents O or S.

Particular preference is given to compounds of the general formula (I) in which
R¹ represents phenyl which may be substituted by fluorine, chlorine, cyano, methyl, ethyl, tert-butyl, methoxy, ethoxy, trifluoromethyl, trifluoromethoxy, amino, dimethylamino or diethylamino,
R² and R³ each represent methyl or together with the carbon atom to which they are attached form a spiro-linked cyclopentane or cyclohexane ring,
R⁴ represents hydrogen,
R⁵ and R⁶ represent hydrogen or together with the carbon atom to which they are attached form a carbonyl group,
R⁷ represents hydrogen, methyl, methoxy, ethoxy, fluorine or chlorine,
R⁸ and R⁹ in each case represent hydrogen,
R¹⁰ represents hydrogen,
X represents O
and
Y represents a bond
or
X represents a bond
and
Y represents O.

The general or preferred radical definitions listed above apply both to the end products of the formula (I) and, correspondingly, to the starting materials and intermediates required in each case for the preparation.

The individual radical definitions given in the respective combinations or preferred combinations of radicals are, independently of the respectively given combinations of radicals, also replaced by any radical definitions of other combinations.

Very particular preference is given to combinations of two or more of the abovementioned preferred ranges.

Of particular importance are compounds of the formula (I-A) 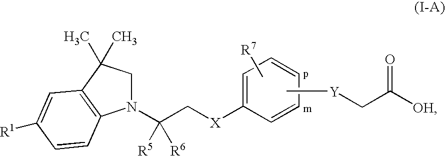
in which
R¹ represents phenyl which is substituted by fluorine, chlorine or trifluoromethyl,
R⁵ and R⁶ represent hydrogen or together with the carbon atom to which they are attached form a carbonyl group,
R⁷ represents hydrogen, methyl, methoxy, ethoxy, fluorine or chlorine,
X represents O
and
Y represents a bond
or
X represents a bond
and
Y represents O,
   and the group attached via Y is located in the para- or meta-position (marked in formula (I-A)) of the phenyl ring relative to the substitutent X.

Moreover, we have found a process for preparing the compounds of the general formula (I) or (I-A) according to the invention, which process is characterized in that compounds of the formula (II) 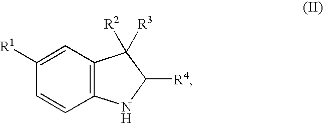
in which R¹, R², R³ and R⁴ are each as defined above
are either
[A] coupled in an inert solvent in the presence of a condensating agent and if appropriate in the presence of an auxiliary base with a compound of the formula (III) 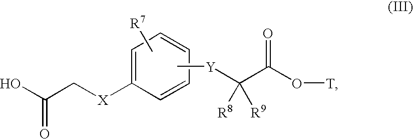
   in which
   X, Y, R⁷, R⁸ and R⁹ are each as defined above and
   T represents benzyl or (C₁-C₆)-alkyl,
   to give compounds of the formula (IV) 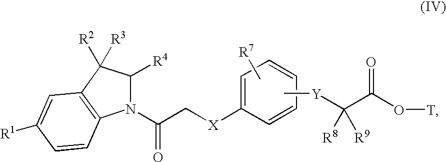
   in which R¹, R², R³, R⁴, R⁷, R⁸, R⁹, X, Y and T are each as defined above,
   which, if R⁵ and R⁶ in formula (I) or (I-A) represent hydrogen are, in an inert solvent in the presence of a suitable reducing agent, converted further into compounds of the formula (V) 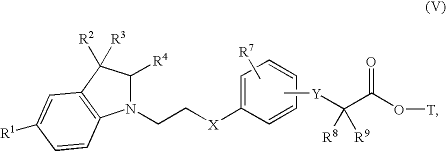
   in which R¹, R², R³, R⁴, R⁷, R⁸, R⁹, X, Y and T are each as defined above,
   the compounds of the formula (IV) or (V) are then converted with acids or bases or, if T represents benzyl, also hydrogenolytically, into the corresponding carboxylic acids of the formula (VI) 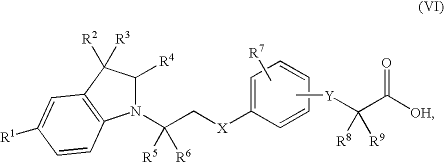
   in which R¹, R², R³, R⁴, R⁵, R⁶, R⁷, R⁸, R⁹, X and Y are each as defined above,
or else
[B] in the case that in formula (I) or (I-A) X represents a bond and Y represents O, S or N—R¹¹, initially coupled in an inert solvent in the presence of a condensating agent and if appropriate in the presence of an auxiliary base with a compound of the formula (VII) 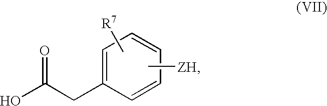
   in which
   R⁷ is as defined above and
   Z represents O, S or N—R¹¹, where R¹¹ is as defined above,
   to give compounds of the formula (VI) 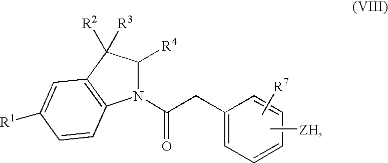
   in which R¹, R², R³, R⁴, R⁷ and Z are each as defined above,
   which are then, in an inert solvent in the presence of a base, reacted with a compound of the formula (IX) 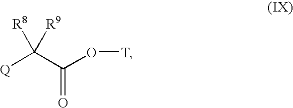
   in which R⁸, R⁹ and T are each as defined above and
   Q represents a suitable leaving group, such as, for example, halogen, mesylate or tosylate,
   to give compounds of the formula (X) 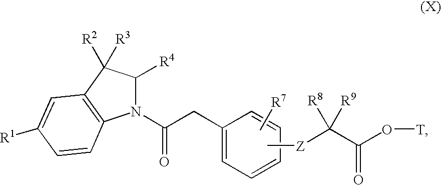
   in which R¹, R², R³, R⁴, R⁷, R⁸, R⁹, T and Z are each as defined above,
   which, if R⁵ and R⁶ in formula (I) or (I-A) represent hydrogen, are, in an inert solvent in the presence of a suitable reducing agent, converted further into compounds of the formula (XI) 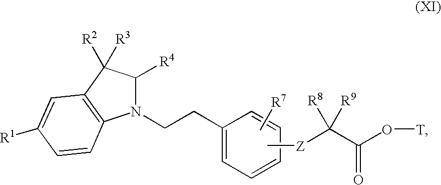
   in which R¹, R², R³, R⁴, R⁷, R⁸, R⁹, T and Z are each as defined above,
   the compounds of the formula (X) or (XI) are then, with acids or bases or, if T represents benzyl, also hydrogenolytically, converted into the corresponding carboxylic acids of the formula (XII) 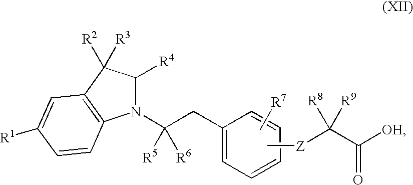
   in which R¹, R², R³, R⁴, R⁵, R⁶, R⁷, R⁸, R⁹ and Z are each as defined above,
the carboxylic acids of the formula (VI) or (XII) are, if appropriate, modified further into compounds of the formula (I) or (I-A) using known esterification methods,
   and the resulting compounds of the formula (VI), (XII), (I) or (I-A) are, if appropriate, converted into their solvates, salts and/or solvates of the salts using the corresponding (i) solvents and/or (ii) bases or acids.

Inert solvents for process step (II)+(III)→(IV) or (II)+(VII)→(VIII) are, for example, ethers, such as diethyl ether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethylene or chlorobenzene, or other solvents, such as ethyl acetate, pyridine, dimethyl sulphoxide, dimethylformamide, N,N′-dimethylpropyleneurea (DMPU), N-methylpyrrolidone (NMP), acetonitrile or acetone. It is also possible to use mixtures of the solvents mentioned. Preference is given to dichloromethane, 1,2-dichloroethane or dimethylformamide.

Suitable condensing agents for the amide formation in process step (II)+(III)→(IV) or (II)+(VII)→(VIII) are, for example, carbodiimides, for example N,N′-diethyl-, N,N′-dipropyl-, N,N′-diisopropyl-, N,N′-dicyclohexylcarbodiimide (DCC), N-(3-dimethylaminoisopropyl)-N′-ethylcarbodiimide hydrochloride (EDC) or carbonyl compounds, such as N,N′-carbonyldiimidazole, or 1,2-oxazolium compounds, such as 2-ethyl-5-phenyl-1,2-oxazolium 3-sulphate or 2-tert-butyl-5-methylisoxazolium perchlorate, or acylamino compounds, such as 2-ethoxy-1-ethoxycarbonyl-1,2-dihydroquinoline, or propanephosphonic anhydride, or isobutyl chloroformate, or bis-(2-oxo-3-oxazolidinyl)phosphoryl chloride or benzotriazolyloxytris(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), if appropriate in combination with further auxiliaries, such as 1-hydroxybenzotriazole or N-hydroxysuccinimide, and suitable bases are alkali metal carbonates, for example sodium carbonate or potassium carbonate or sodium bicarbonate or potassium bicarbonate, or organic bases, such as trialkylamines, for example triethylamine, N-methylmorpholine, N-methylpiperidine or diisopropylethylamine. Preference is given to using EDC.

The process step (II)+(III)→(IV) or (II)+(VII)→(VI) is generally carried out in a temperature range of from 0° C. to +100° C., preferably from 0° C. to +40° C. The reaction can be carried out under atmospheric, elevated or reduced pressure (for example from 0.5 to 5 bar). In general, the reaction is carried out under atmospheric pressure. Suitable reducing agents for process step (IV)→(V) or (X)→(XI) are, for example, bocrude ydrides, such as borane or diborane, including the complexes, for example with tetrahydrofuran or dimethyl sulphide, or else diphenylsilane in the presence of carbonyl-tris(triphenylphosphine)rhodium(I) hydride as catalyst [see R. Kuwano, M. Takahashi, Y. Ito, Tetrahedron Lett. 1998, 39, 1017-1020].

Inert solvents for process step (IV)→(V) or (X)→(XI) are, for example, ethers, such as diethyl ether, dioxane, tetrahydrofuran, glycol dimethyl ether or diethylene glycol dimethyl ether, or hydrocarbons, such as benzene, toluene, xylene, hexane, cyclohexane or mineral oil fractions. It is also possible to use mixtures of the solvents mentioned. Preference is given to tetrahydrofuran.

The reaction is generally carried out in a temperature range of from −20° C. to +80° C., preferably from 0° C. to +40° C. The reaction can be carried out under atmospheric, elevated or reduced pressure (for example from 0.5 to 5 bar). In general, the reaction is carried out under atmospheric pressure.

Inert solvents for process step (VIII)+(IX)→(X) are, for example, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as acetone, 2-butanone, dimethylformamide, dimethyl sulphoxide, acetonitrile or N-methylpyrrolidinone. It is also possible to use mixtures of the solvents mentioned. Preference is given to dimethylformamide or acetone.

Suitable bases for process step (VII)+(IX)→(X) are the customary inorganic or organic bases. These include alkali metal hydroxides, such as, for example, lithium hydroxide, sodium hydroxide or potassium hydroxide, alkali metal or alkaline earth metal carbonates, such as sodium carbonate, potassium carbonate or calcium carbonate, alkali metal hydrides, such as sodium hydride, or organic amines, such as pyridine, triethylamine, ethyldiisopropylamine, N-methylmorpholine or N-methylpiperidine. Preference is given to potassium carbonate or sodium hydride.

Here, the base is employed in an amount of from 1 to 5, preferably from 1 to 2, mol, based on 1 mol of the compound of the formula (VI).

The reaction is generally carried out in a temperature range of from −20° C. to +150° C., preferably from 0° C. to +80° C. The reaction can be carried out under atmospheric, elevated or reduced pressure (for example from 0.5 to 5 bar). In general, the reaction is carried out under atmospheric pressure.

Inert solvents for process step (IV)/(V)→(VI) or (X)/(XI)→(XII) are, for example, halogenated hydrocarbons, such as dichloromethane, 1,2-dichloroethane or trichloroethylene, ethers, such as diethyl ether, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as nitromethane, acetone, dimethylformamide, dimethyl sulphoxide, acetonitrile, N-methylpyrrolidinone or else water. It is also possible to use mixtures of the solvents mentioned. In the case of basic ester hydrolysis, preference is given to alcohols, such as methanol or ethanol, and mixtures thereof with tetrahydrofuran, and in the case of an acidic ester cleavage, preference is given to dichloromethane.

Suitable bases for process step (IV)/(V)→(VI) or (X)/(XI)→(XII) are the customary inorganic bases. These preferably include alkali metal hydroxides, such as, for example, lithium hydroxide, sodium hydroxide or potassium hydroxide, or alkali metal or alkaline earth metal carbonates, such as sodium carbonate, potassium carbonate or calcium carbonate. Particular preference is given to lithium hydroxide or sodium hydroxide.

Here, the base is employed in an amount of from 1 to 5, preferably from 1 to 3, mol, based on 1 mol of the compound of the formula (IV), (V), (X) or (XI).

Suitable acids for process step (IV)/(V)→(VI) or (X)/(XI)→(XII) are the customary inorganic acids, such as, for example, hydrochloric acid or sulphuric acid, or sulphonic acids, such as toluenesulphonic acid, methanesulphonic acid or trifluoromethanesulphonic acid, or carboxylic acids, such as trifluoroacetic acid.

The reaction is generally carried out in a temperature range of from −20° C. to +100° C., preferably from 0° C. to +30° C. The reaction can be carried out under atmospheric, elevated or reduced pressure (for example from 0.5 to 5 bar). In general, the reaction is carried out under atmospheric pressure.

The compounds of the formula (II) can be prepared analogously to processes known from the literature by reacting compounds of the formula (XII) 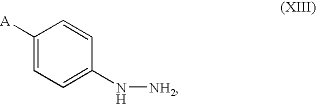
in which
A represents chlorine or bromine
   in the presence of an acid or Lewis acid, if appropriate in an inert solvent, with a compound of the formula (XIV) 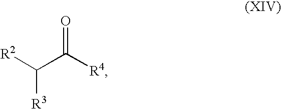
   in which R², R³ and R⁴ are each as defined above,
   if R² and R³ in (XIV) are both not hydrogen, to give compounds of the formula (XV), or, if R³ in (XIV) is hydrogen, to give compounds of the formula (XVI) 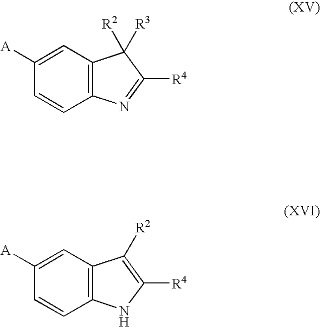
   in which A and R⁴ are each as defined above,
   then reducing the compounds of the formula (XV) or (XVI) with the aid of a boron, aluminium or silicon hydride, such as, for example, sodium bocrude ydride or sodium cyano-bocrude ydride, or by hydrogenation in the presence of a suitable catalyst, such as, for example, Raney nickel, to give compounds of the formula (XVII) 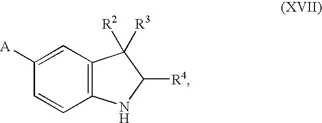
   in which A, R², R³ and R⁴ are each as defined above
   [for process steps (XIII)+(XIV)→(XV)→(XVII) cf., for example, P. E. Maligres, I. Houpis, K. Rossen, A. Molina, J. Sager, V. Upadhyay, K. M. Wells, R. A. Reamer, J. E. Lynch, D. Askin, R. P. Volante, P. J. Reider, Tetrahedron 1997, 53, 10983-10992], then converting the compounds of the formula (XVII) by methods known from the literature into compounds of the formula (XVIII) 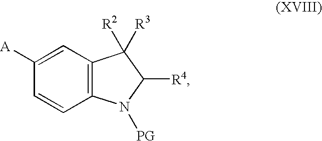
   in which A, R², R³ and R⁴ are each as defined above and
PG represents a suitable amino protective group, preferably 4-nitrophenylsulphonyl,
   then reacting these in a coupling reaction with a compound of the formula (XIX) 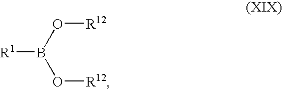
   in which R¹ is as defined above and
R¹² represents hydrogen or methyl or both radicals together form a CH₂CH₂— or C(CH₃)₂—C(CH₃)₂— bridge,
   in an inert solvent in the presence of a suitable palladium catalyst and a base to give compounds of the formula (XX) 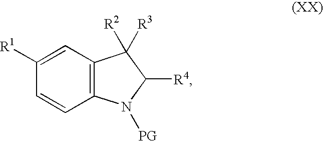
   in which PG, R¹, R², R³ and R⁴ are each as defined above
   [cf, for example, W. Hahnfeld, M. Jung, Pharmacie 1994, 49, 18-20; idem, Liebigs Ann. Chem. 1994, 59-64] and finally removing the protective group PG by methods known from the literature to give compounds of the formula (II).

Inert solvents for process step (XIII)+(XIV)→(XV) or (XVI) are, for example, halogenated hydrocarbons, such as dichloromethane, trichloromethane, carbon tetrachloride, trichloroethane, tetrachloroethane, 1,2-dichloroethane or trichloroethylene, ethers, such as dioxane, tetrahydrofuran, glycol dimethyl ether or diethylene glycol dimethyl ether, alcohols, such as methanol, ethanol, n-propanol, isopropanol, n-butanol or tert-butanol, or hydrocarbons, such as benzene, xylene, toluene, hexane, cyclohexane or mineral oil fractions, or other solvents, such as acetonitrile or water. It is also possible to use mixtures of the solvents mentioned. It is also possible to carry out the reaction in the absence of a solvent. If R³ represents hydrogen, the reaction is preferably carried out in the absence of a solvent to give the product (XVI), if R² and R³ are both not hydrogen, the reaction is preferably carried out in a mixture of toluene and acetonitrile to give the product (XV).

Suitable acids for process step (XIII)+(XIV)→(XV) or (XVI) are the customary inorganic or organic acids. These preferably include hydrochloric acid, sulphuric acid or phosphoric acid, or carboxylic acids, such as formic acid, acetic acid or trifluoroacetic acid, or sulphonic acids, such as toluenesulphonic acid, methanesulphonic acid or trifluoromethanesulphonic acid. Alternatively, the customary Lewis acids, such as, for example, boron trifluoride, aluminium trichloride or zinc chloride are also suitable. Here, the acid is employed in an amount of from 1 to 10 mol, based on 1 mol of the compound of the formula (XIII). If R³ represents hydrogen, the reaction is preferably carried out using 1 to 2 mol of zinc chloride to give the product (XVI), and if R² and R³ are both not hydrogen, the reaction is preferably carried out using 2 to 5 mol of trifluoroacetic acid to give the product (XV).

The reaction is generally carried out in a temperature range of from 0° C. to +250° C. If R³ represents hydrogen, the reaction is preferably carried out in a temperature range of from +130° C. to +200° C. to give the product (XVI), if R² and R³ are both not hydrogen, the reaction is preferably carried out in a temperature range of from 0° C. to +50° C. to give the product (XV). The reaction can be carried out under atmospheric, elevated or reduced pressure (for example from 0.5 to 5 bar). In general, the reaction is carried out under atmospheric pressure.

Reducing agents suitable for process step (XV) or (XVI)→(XVII) are boron hydrides, aluminium hydrides or silicon hydrides, such as, for example, borane, diborane, sodium borohydride, sodium cyanoborohydride, lithium aluminium hydride or triethylsilane, if appropriate in the presence of an acid or Lewis acid, such as, for example, acetic acid, trifluoroacetic acid, aluminium trichloride or boron trifluoride, or the hydrogenation with hydrogen in the presence of a suitable catalyst, such as, for example, palladium-on-carbon, platinum oxide or Raney nickel. In the case of compounds of the formula (XVI), preference is given to the reduction using sodium cyanoborohydride; in the case of compounds of the formula (XV), preference is given to using sodium borohydride.

Suitable solvents for process step (XV) or (XVI)→(XVII) are, for example, ethers, such as diethyl ether, dioxane, tetrahydrofuran, glycol dimethyl ether or diethylene glycol dimethyl ether, alcohols, such as methanol, ethanol, n-propanol, isopropanol, n-butanol or tert-butanol, or hydrocarbons, such as benzene, xylene, toluene, hexane, cyclohexane or mineral oil fractions, or other solvents, such as acetonitrile, acetic acid or water. It is also possible to use mixtures of the solvents mentioned. For the reduction of the compounds of the formula (XVI), preference is given to using acetic acid, a large excess of which, added as acid to the reducing agent, simultaneously serves as solvent. For the reduction of the compounds of the general formula (XV), preference is given to using a mixture of methanol and toluene/acetonitrile [from the reaction (XIII)→(XV), with the addition of 2 to 5 mol of trifluoroacetic acid] in a ratio of from 1:1 to 1:10.

The reaction is generally carried out in a temperature range of from −20° C. to +100° C., preferably from −10° C. to +50° C. The reaction can be carried out under atmospheric, elevated or reduced pressure (for example from 0.5 to 5 bar). In general, the reaction is carried out under atmospheric pressure.

Inert solvents for process step (XVIII)+(XIX)→(XX) are, for example, ethers, such as diethyl ether, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as dimethylformamide, acetonitrile or else water. It is also possible to use mixtures of the solvents mentioned. Preference is given to toluene, dimethylformamide or acetonitrile.

Suitable bases for process step (XVIII)+(XIX)→(XX) are the customary inorganic or organic bases. These preferably include alkali metal hydroxides, such as, for example, lithium hydroxide, sodium hydroxide or potassium hydroxide, alkali metal or alkaline earth metal carbonates, such as sodium carbonate, potassium carbonate or calcium carbonate, alkali metal phosphates, such as sodium phosphate or potassium phosphate, or organic amines, such as pyridine, triethylamine, ethyldiisopropylamine, N-methylmorpholine or N-methylpiperidine. Particular preference is given to sodium carbonate or potassium carbonate or potassium phosphate.

Here, the base is employed in an amount of from 1 to 5, preferably from 2 to 3, mol, based on 1 mol of the compound of the formula (XVII).

Suitable palladium catalysts for process step (XVIII)+(XIX)→(XX) are, preferably, palladium(0) or palladium(II) compounds which are used pre-formed, such as, for example, [1,1′-bis(diphenylphosphino)ferrocenyl]palladium(II) chlorine, bis(triphenylphosphine)palladium(II) chloride or tetrakis(triphenylphosphine)palladium(0), or those which can be generated in situ from a suitable palladium source, such as, for example, bis(dibenzylideneacetone)palladium(0) and a suitable phosphine ligand.

The reaction is generally carried out in a temperature range of from 0° C. to +150° C., preferably from +20° C. to +120° C. The reaction can be carried out under atmospheric, elevated or reduced pressure (for example from 0.5 to 5 bar). In general, the reaction is carried out under atmospheric pressure.

The compounds of the formula (III) can be prepared analogously to processes known from the literature, for example by reacting compounds of the formula (XXI) 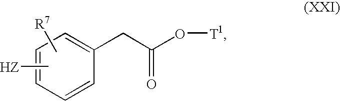
in which R⁷ and Z are each as defined above and
T¹ represents benzyl or (C₁-C₆)-alkyl
   in an inert solvent in the presence of a base with a compound of the formula (XXII) 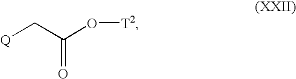
   in which Q is as defined above and
T² represents benzyl or (C₁-C₆)-alkyl, but its specific meaning is different from that of T¹,
   to give compounds of the formula (XXIII) 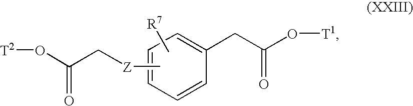
   in which R⁷, Z, T¹ and T² are each as defined above
   and then optionally converting these under controlled chemoselective reaction conditions with acids or bases or, if T¹ or T² represents benzyl, also hydrogenolytically into the corresponding carboxylic acids of the formula (XXIV) or (XXV) 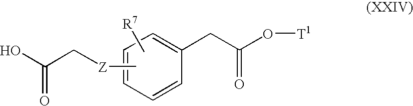 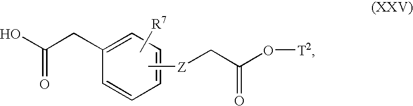
   respectively, in which R⁷, Z and T¹ or T², respectively, are each as defined above.

Inert solvents for process step (XXI)+(XXII)→(XXIII) are, for example, ethers, such as diethyl ether, dioxane, tetrahydrofuran, glycol dimethyl ether or diethylene glycol dim ethyl ether, hydrocarbons, such as benzene, xylene, toluene, hexane, cyclohexane or mineral oil fractions, or other solvents, such as acetone, 2-butanone, dimethylformamide, dimethyl sulphoxide, acetonitrile or N-methylpyrrolidinone. It is also possible to use mixtures of the solvents mentioned. Preference is given to dimethylformamide or acetone.

Suitable bases for process step (XXI)+(XXII)→(XXIII) are the customary inorganic or organic bases. These include alkali metal hydroxides, such as, for example, lithium hydroxide, sodium hydroxide or potassium hydroxide, alkali metal or alkaline earth metal carbonates, such as sodium carbonate, potassium carbonate or calcium carbonate, alkali metal hydrides, such as sodium hydride, or organic amines, such as pyridine, triethylamine, ethyldiisopropylamine, N-methylmorpholine or N-methylpiperidine. Preference is given to potassium carbonate or sodium hydride.

Here, the base is employed in an amount of from 1 to 5, preferably from 1 to 2, mol, based on 1 mol of the compound of the formula (XXI).

The reaction is generally carried out in a temperature range of from −20° C. to +150° C., preferably from 0° C. to +80° C. The reaction can be carried out under atmospheric, elevated or reduced pressure (for example from 0.5 to 5 bar). In general, the reaction is carried out under atmospheric pressure.

Inert solvents for process step (XXIII)→(XXIV) or (XXV) are, for example, halogenated hydrocarbons, such as dichloromethane, 1,2-dichloroethane or trichloroethylene, ethers, such as diethyl ether, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as nitromethane, acetone, dimethylformamide, dimethyl sulphoxide, acetonitrile, N-methylpyrrolidinone or else water. It is also possible to use mixtures of the solvents mentioned. In the case of a basic ester hydrolysis, preference is given to alcohols, such as methanol or ethanol and mixtures thereof with tetrahydrofuran, and in the case of an acidic ester cleavage, preference is given to dichloromethane.

Suitable bases for process step (XXIII)→(XXIV) or (XXV) are the customary inorganic bases. These preferably include alkali metal hydroxides, such as, for example, lithium hydroxide, sodium hydroxide or potassium hydroxide, or alkali metal or alkaline earth metal carbonates, such as sodium carbonate, potassium carbonate or calcium carbonate. Particular preference is given to lithium hydroxide or sodium hydroxide.

Here, the base is employed in an amount of from 1 to 5, preferably from 1 to 3, mol, based on 1 mol of the compound of the formula (XXIII).

Suitable acids for process step (XXIII)→(XXIV) or (XXV) are the customary inorganic acids, such as, for example, hydrochloric acid or sulphuric acid, or sulphonic acids, such as toluenesulphonic acid, methanesulphonic acid or trifluoromethanesulphonic acid, or carboxylic acids, such as trifluoroacetic acid.

The reaction is generally carried out in a temperature range of from −20° C. to +100° C., preferably from 0° C. to +30° C. The reaction can be carried out under atmospheric, elevated or reduced pressure (for example from 0.5 to 5 bar). In general, the reaction is carried out under atmospheric pressure.

The compounds of the formulae (VII), (IX), (XIII), (XIV), (XIX), (XXI) and (XXII) are commercially available, known from literature or can be prepared analogously to processes known from the literature.

The process according to the invention can be illustrated by reaction schemes 1 to 3 below:

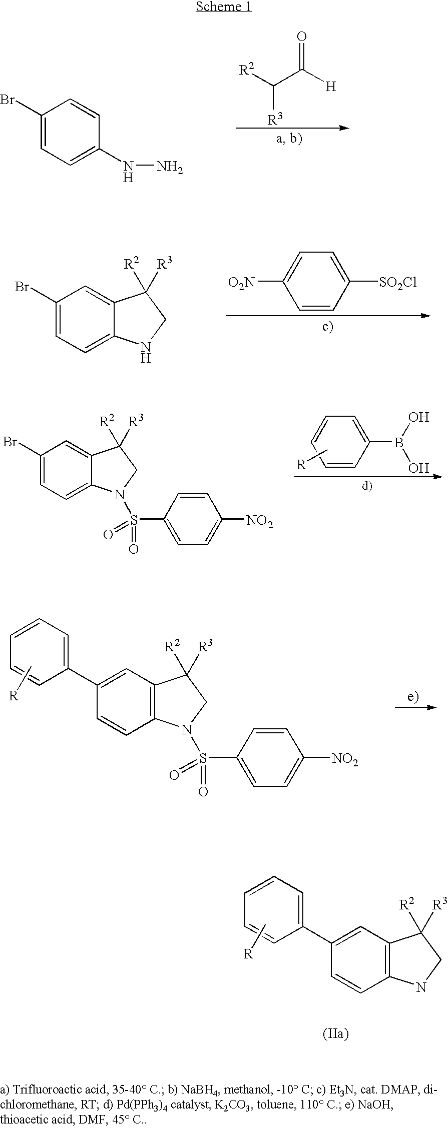

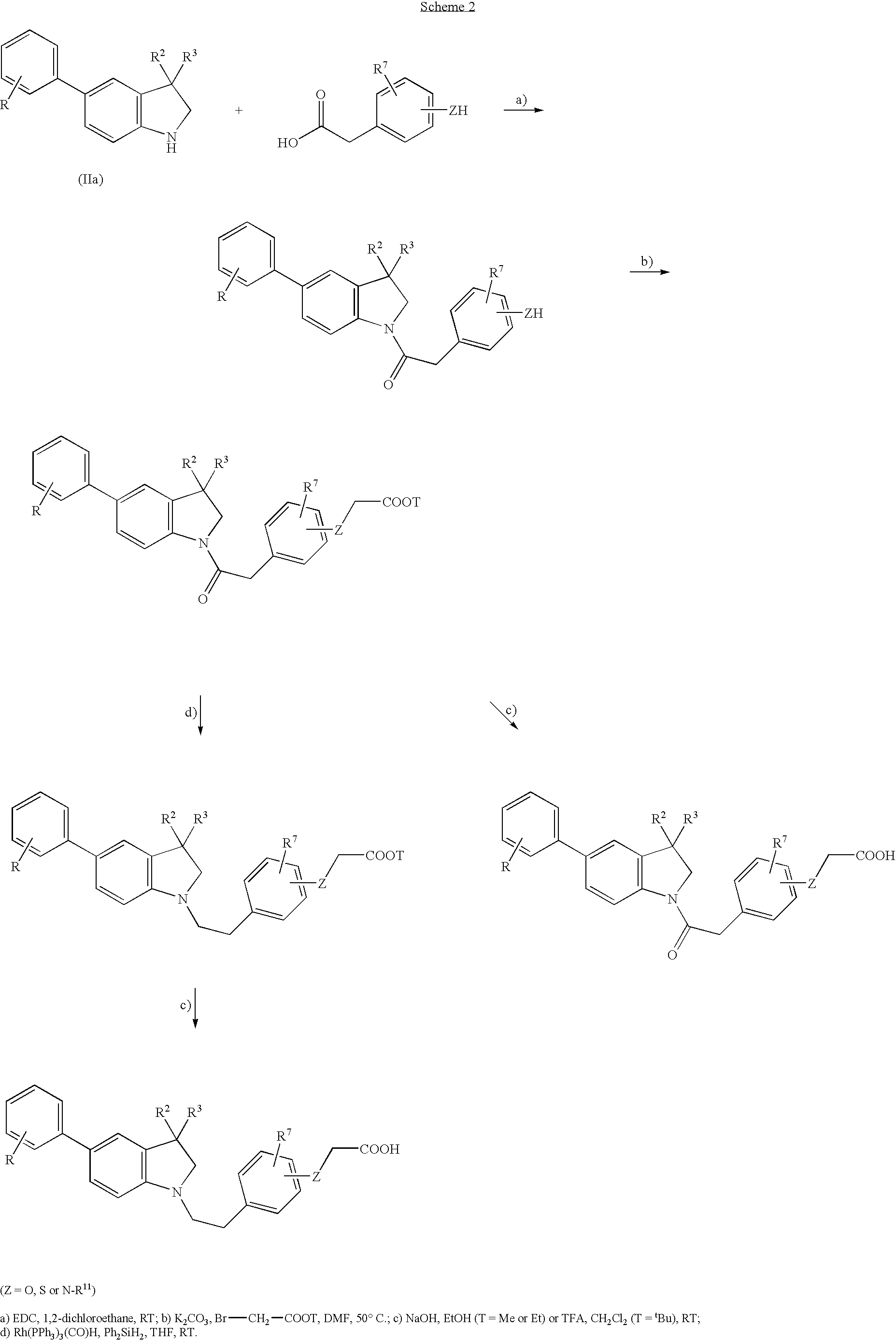

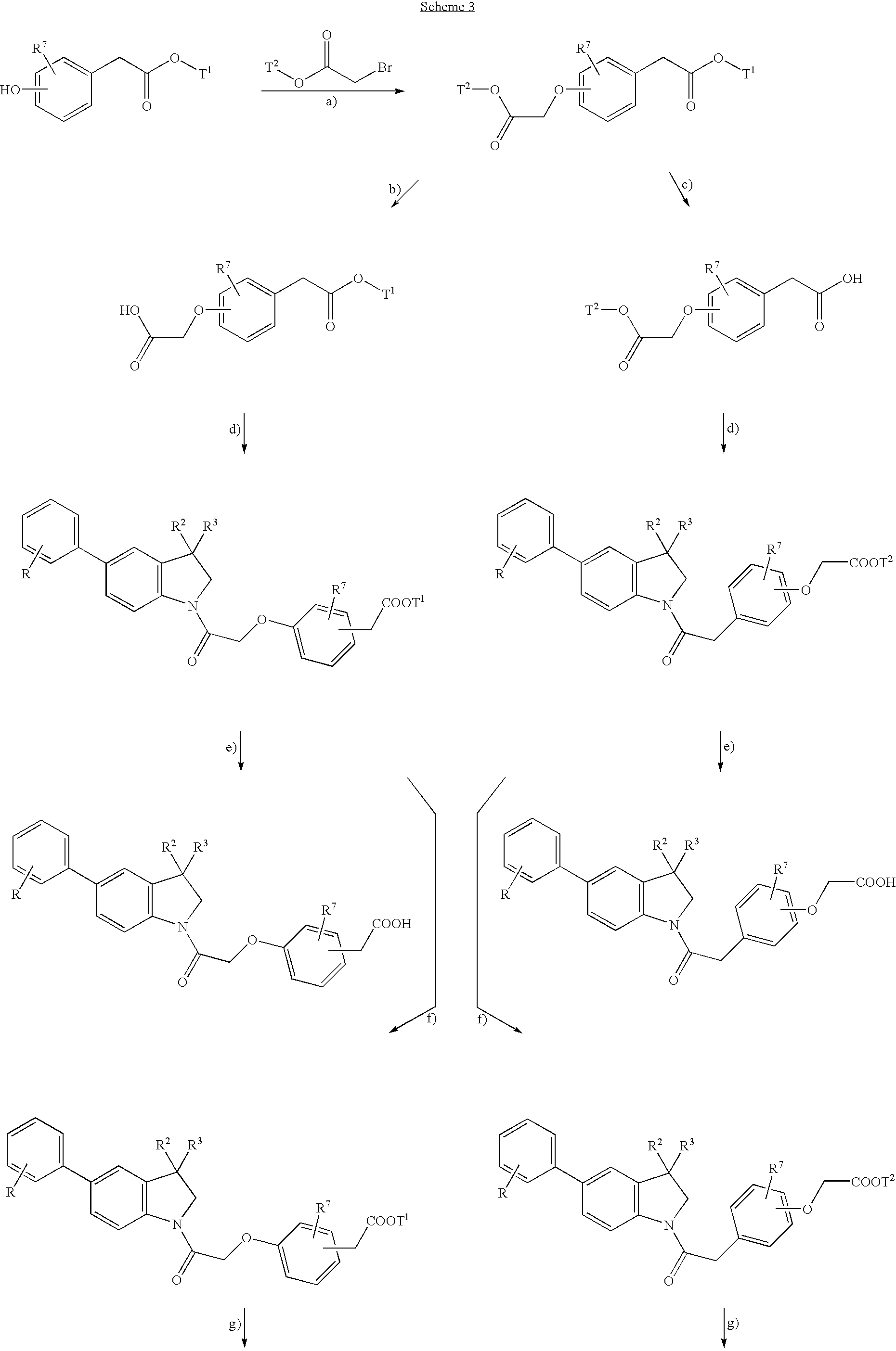

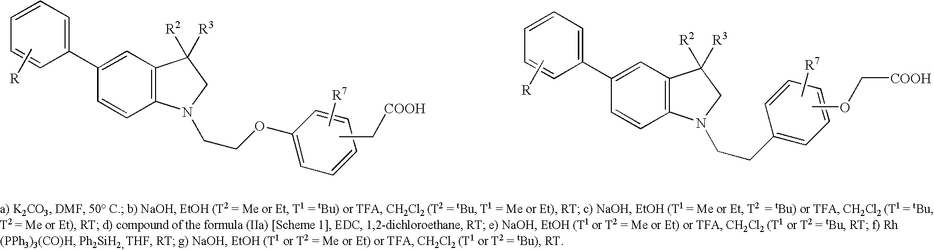

The compounds of the formula (I and (I-A) according to the invention have a surprising and useful spectrum of pharmacological activity and can therefore be used as versatile medicaments. In particular, they are suitable for treating coronary heart disease, for the prophylaxis of myocardial infarction and for the treatment of restenosis after coronary angioplasty or stenting. The compounds of the formula (I) and (I-A) according to the invention are preferably suitable for treating arteriosclerosis and hypercholesterolaemia, for increasing pathologically low HDL levels and for lowering elevated triglyceride and LDL levels. In addition, they can be used for treating obesity, diabetes, for treating metabolic syndrome (glucose intolerance, hyperinsulinaemia, dyslipidaemia and high blood pressure owing to insulin resistance), hepatic fibrosis and cancer.

The novel active compounds can be administered alone or, if required, in combination with other active compounds, preferably from the group of the CETP inhibitors, antidiabetics, antioxidants, cytostatics, calcium antagonists, antihypertensives, thyroid hormones and/or thyroid mimetics, inhibitors of HMG-CoA reductase, inhibitors of HMG-CoA reductase expression, squalene synthesis inhibitors, ACAT inhibitors, perfusion promoters, platelet aggregation inhibitors, anticoagulants, angiotensin II receptor antagonists, cholesterol absorption inhibitors, MTP inhibitors, aldolase reductase inhibitors, fibrates, niacin, anorectics, lipase inhibitors and PPAR-α and/or PPAR-γ agonists.

The activity of the compounds according to the invention can be examined, for example, in vitro by the transactivation assay described in the experimental section.

The activity of the compounds according to the invention in vivo can be examined, for example, by the tests described in the experimental section.

Suitable administration forms for administering the compounds of the general formula (I) and (I-A) are all customary administration forms, i.e. oral, parenteral, inhalative, nasal, sublingual, rectal, external, for example transdermal, or focal, such as, for example, in the case of implants or stents. In the case of parenteral administration, particular mention has to be made of intravenous, intramuscular and subcutaneous administration, for example as a subcutaneous depot. Preference is given to oral or parenteral administration. Very particular preference is given to oral administration.

Here, the active compounds can be administered on their own or in the form of preparations. Preparations suitable for oral administration are, inter alia, tablets, capsules, pellets, sugar-coated tablets, pills, granules, solid and liquid aerosols, syrups, emulsions, suspensions and solutions. Here, the active compound has to be present in such an amount that a therapeutic effect is obtained. In general, the active compound can be present in a concentration of from 0.1 to 100% by weight, in particular from 0.5 to 90% by weight, preferably from 5 to 80% by weight. In particular, the concentration of active compound should be 0.5-90% by weight, i.e. the active compound should be present in amounts sufficient to reach the dosage range stated.

To this end, the active compounds can be converted in a manner known per se into the customary preparations. This is carried out using inert non-toxic pharmaceutically acceptable carriers, auxiliaries, solvents, vehicles, emulsifiers and/or dispersants.

Auxiliaries which may be mentioned are, for example: water, non-toxic organic solvents, such as, for example, paraffins, vegetable oils (for example sesame oil), alcohols (for example ethanol, glycerol), glycols (for example polyethylene glycol), solid carriers, such as natural or synthetic ground minerals (for example talc or silicates), sugar (for example lactose), emulsifiers, dispersants (for example polyvinylpyrrolidone) and glidants (for example magnesium sulphate).

In the case of oral administration, tablets may, of course, also contain additives such as sodium citrate, together with additives such as starch, gelatine and the like. Aqueous preparations for oral administration may furthermore comprise flavour improvers or colorants.

In the case of oral administration, preference is given to administering dosages of from 0.001 to 5 mg/kg, preferably from 0.005 to 3 mg/kg, of body weight per 24 hours.

The working examples below illustrate the invention. The invention is not limited to the examples.

In the tests and examples below, the percentages are, unless indicated otherwise, percent by weight; parts are parts by weight. Solvent ratios, dilution ratios and concentrations of liquid/liquid solutions are in each case based on volume.

### A. EXAMPLES

Abbreviations:

^{t}Bu tert-butyl

DCI direct chemical ionization (in MS)

DMAP 4-N,N-dimethylaminopyridine

DMF N,N-dimethylformamide

DMSO dimethyl sulphoxide

EDC N′-(3-dimethylaminopropyl)-N-ethylcarbodiimide×HCl

ESI electrospray Ionization (in MS)

Et Ethyl

h hour(s)

HPLC high pressure, high performance liquid chromatography

LC/MS liquid chromatography-coupled mass spectroscopy

Me methyl

min minute(s)

MS mass spectroscopy

NMR nuclear magnetic resonance spectroscopy

Ph phenyl

R_{f} retention index (in TLC)

RT room temperature

TFA trifluoroacetic acid

THF tetrahydrofuran

UV ultraviolet spectrum

HPLC and LC/MS Methods:

Method 1:

Instrument: Micromass Platform LCZ with HPLC Agilent Series 1100; column: Grom-Sil 120 ODS-4 HE, 50 mm×2.0 mm, 3 μm; mobile phase A: 1 l of water+1 ml of 50% strength formic acid, mobile phase B: 1 l of acetonitrile+1 ml of 50% strength formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 208-400 nm.

Method 2:

Instrument: HP 1100 with DAD detection; column: Kromasil RP-18, 60 mm×2 mm, 3.5 μm; mobile phase A: 5 ml of HClO₄/l of water, mobile phase B: acetonitrile; gradient: 0 min 2% B→0.5 min 2% B→4.5 min 90% B→9 min 90% B; flow rate: 0.75 ml/min; oven: 30° C.; UV detection: 210 nm.

Method 3:

Instrument: HP 1100 with DAD detection; column: Kromasil RP-18, 60 mm×2 mm, 3.5 μm; mobile phase A: 5 ml of HClO₄/l of water, mobile phase B: acetonitrile; gradient: 0 min 2% B<0.5 min 2% B→4.5 min 90% B→6.5 min 90% B; flow rate: 0.75 ml/min; oven: 30° C.; UV detection: 210 nm.

Method 4:

Instrument: HP 1100 with DAD detection; column: Kromasil RP-18, 125 mm×2 mm, 3.5 μm; mobile phase A: PIC B7-heptanesulphonic acid (from Waters, art. no. WAT084282), mobile phase B: acetonitrile; gradient: 0 min 2% B<1 min 2% B→9 min 90% B→13 min 90% B; flow rate: 2 ml/min; oven: 30° C.; UV detection: 210 nm.

Method 5:

MS-instrument: Micromass ZQ; HPLC-instrument: Waters Alliance 2790; column: Grom-Sil 120 ODS-4 HE, 50 mm×2 mm, 3.0 μm; mobile phase A: water-+500 μl of 50% strength formic acid/l, mobile phase B: acetonitrile+500 μl of 50% strength formic acid/l; gradient: 0.0 min 5% B→2.0 min 40% B→4.5 min 90% B→5.5 min 90% B; oven: 45° C.; flow rate: 0.0 min 0.75 ml/min→4.5 min 0.75 ml/min→5.5 min 1.25 ml/min; UV detection: 210 mm.

Method 6:

Instrument: Micromass Quattro LCZ, with HPLC Agilent series 1100; column: Grom-Sil 120 ODS-4 HE, 50 mm×2.0 mm, 3 μm; mobile phase A: 1 l of water+1 ml of 50% strength formic acid, mobile phase B: 1 l of acetonitrile+1 ml of 50% strength formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 208-400 nm.

Starting Materials:

### Example 1A

### 5-Bromo-3,3-dimethylindoline

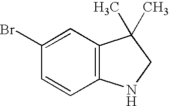

A mixture of 45 ml of toluene/acetonitrile (49:1) is flushed with argon for 5 minutes, and 3.00 g (16.0 mmol) of 4-bromophenylhydrazine are then added. 3.71 ml (48.1 mmol) of trifluoroacetic acid are then added slowly, and it is made sure that a temperature of 35° C. is not exceeded. The temperature is then maintained at 35° C., and over a period of 2 h a solution of 1.05 g (14.6 mmol) of isobutyraldehyde in 4 ml of toluene/acetonitrile (49:1) is slowly added dropwise. The mixture is stirred at 35° C. for 4 h and at room temperature for 2 h. The mixture is then cooled to −10° C., 4.0 ml of methanol are added and 819 mg (21.7 mmol) of solid sodium borohydride is added a little at a time over a period of 30 min. During the addition, the temperature must not exceed −2° C. After the addition has ended, the mixture is stirred at 0° C. for 1 h. 150 ml of a 6% by weight strength solution of ammonia in water are added, and the phases are then separated and in each case 1.5 ml of acetonitrile and methanol are added to the organic phase. The organic phase is then washed with 150 ml of a 15% strength solution of sodium chloride in water and dried over sodium sulphate. The mixture is filtered through 100 g of silica gel and washed twice with in each case 200 ml of diethyl ether. The organic filtrate is concentrated under reduced pressure and chromatographed on 100 g of silica gel. Initially, the byproducts are eluted using cyclohexane, and the desired product is then eluted using a mixture of cyclohexane/diethyl ether (20:1).

Yield: 1.78 g (54% of theory)

R_{f} (petroleum ether/ethyl acetate 5:1)=0.47

UV [nm]=200, 268, 276

MS (ESIpos): m/z=226 [M+H]⁺

¹H-NMR (200 MHz, DMSO-d₆): δ=1.20 (s, 6H), 3.18 (d, 2H), 5.66 (br. s, 1H), 6.42 (d, 1H), 7.02 (dd, 1H), 7.10 (d, 1H).

### Example 2A

### 5-Bromo-3,3-dimethyl-1-(4-nitrobenzene)sulphonylindoline

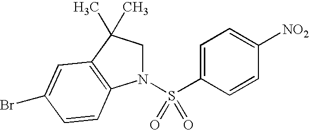

At 5-10° C., a solution of 17.50 g (78.94 mmol) of 4-nitrobenzenesulphonyl chloride in 150 ml of dichloromethane is added dropwise to a solution of 17.00 g (75.18 mmol) of 5-bromo-3,3-dimethylindoline, 0.46 g (3.76 mmol) of DMAP and 21 ml (15.22 g, 150.4 mmol) of triethylamine in 100 ml of dichloromethane. The mixture is then stirred at room temperature overnight. 2 N aqueous hydrochloric acid is added to the reaction mixture, the phases are separated and the organic phase is washed with water and saturated sodium chloride solution. Drying over sodium sulphate and removal of the solvent under reduced pressure gives 31 g (98% of theory) of the desired product.

HPLC (method 1): Rₜ=4.1 min.

MS (DCI): m/z=428 [M+NH₄]⁺

¹H-NMR (300 MHz, CDCl₃): δ=1.14 (s, 6H), 3.68 (s, 2H), 7.15 (d, 1H), 7.34 (dd, 1H), 7.51 (d, 1H), 8.00 (d, 2H), 8.32 (d, 2H).

### Example 3A

### 3,3-Dimethyl-1-(4-nitrobenzene)sulphonyl-5-(4-trifluoromethyl)phenylindoline

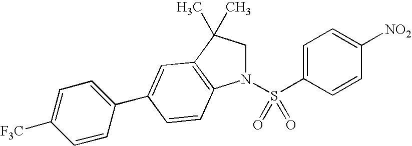

A stream of argon is passed through a suspension of 31.00 g (75.38 mmol) of 5-bromo-3,3-dimethyl-1-(4-nitrobenzene)sulphonylindoline, 21.47 g (113.06 mmol) of 4-trifluoromethylphenylboronic acid and 15.63 g (113.06 mmol) of potassium carbonate in 500 ml of toluene for 15 minutes. 1.72 g (1.51 mmol) of tetrakis(triphenylphosphine)palladium are then added, and the reaction mixture is heated under reflux overnight. After cooling, the mixture is filtered through about 1000 ml of silica gel 60, and the column is then washed with about 1.5 l of cyclohexane and with 2 l of dichloromethane. Removal of the solvent of the dichloromethane fraction under reduced pressure gives 30 g (84% of theory) of the desired product.

HPLC (method 2): Rₜ=5.71 min.

MS (DCI): m/z=494 [M+NH₄]⁺

¹H-NMR (300 MHz, CDCl₃): δ=1.21 (s, 6H), 3.72 (s, 2H), 7.26 (s, 1H), 7.47 (dd, 1H), 7.60 (d, 2H), 7.68 (d, 2H), 7.70 (d, 1H), 8.06 (d, 2H), 8.32 (d, 2H).

### Example 4A

### 3,3-Dimethyl-5-(4-trifluoromethyl)phenylindoline

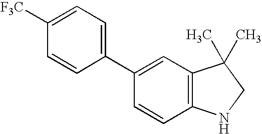

At room temperature, 27.2 ml (28.9 g, 314.0 mmol) of thioacetic acid are quickly added dropwise with stirring to a mixture of 68 g (142.7 mmol) of 3,3-dimethyl-1-(4-nitrobenzene)sulphonyl-5-(4-trifluoromethyl)phenylindoline and 25.1 g (627.9 mmol) of sodium hydroxide in 300 ml of N,N-dimethylformamide. The reaction mixture is then stirred at 45° C. for 5 hours, after cooling, 1 l of ethyl acetate is added and the organic phase is washed twice with saturated sodium carbonate solution and once with saturated sodium chloride solution. After drying over sodium sulphate, the solvent is removed under reduced pressure and the resulting crude product is purified on 1 kg of silica gel 60 (mobile phase cyclohexane/ethyl acetate 7:1). The eluate is concentrated, and the resulting residue is crystallized. This gives 27.1 g (61% of theory) of the desired product.

HPLC (Method 3): Rₜ=4.46 min.

MS (ESIpos): m/z=292 [M+H]⁺

¹H-NMR (300 MHz, CDCl₃): δ=1.37 (s, 6H), 3.40 (s, 2H), 6.70 (d, 1H), 7.27 (d, 1H), 7.30 (dd, 1H), 7.62 (s, 4H).

### Example 5A

### Ethyl [4-(2-tert-butoxy-2-oxoethoxy)-3-ethoxyphenyl]acetate

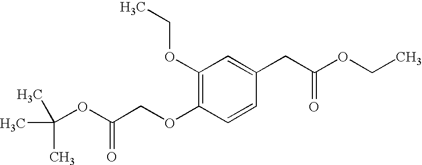

3.39 g (24.526 mmol) of potassium carbonate are added to a solution of 5.0 g (22.3 mmol) of ethyl (3-ethoxy-4-hydroxyphenyl)acetate in 75 ml of DMF, and the mixture is then stirred at 50° C. for 1 h. 3.62 ml (24.53 mmol) of tert-butyl bromoacetate are then added, and and the reaction mixture is stirred at 50° C. overnight. After the reaction has ended, the solvent is distilled off under reduced pressure and the residue is taken up in 150 ml of ethyl acetate and washed three times with in each case 75 ml of water. The organic phase is dried over sodium sulphate and concentrated. This gives 7.5 g (99.4% of theory) of the desired product.

HPLC (Method 3): Rₜ 4.96 min.

MS (DCI): m/z=356 [M+NH₄]⁺

¹H-NMR (300 MHz, CDCl₃): δ=1.22 (t, 3H), 1.42 (t, 3H), 1.48 (s, 9H), 3.5 (s, 2H), 4.12 (tt, 4H), 4.55 (s, 2H), 6.75 (d, 2H), 6.85 (s, 1H).

### Example 6A

### [2-Ethoxy-4-(2-ethoxy-2-oxoethyl)phenoxy]acetic acid

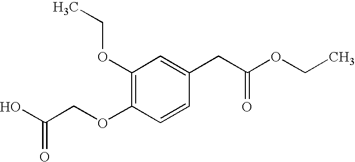

At RT, 7.7 ml (100.0 mmol) of trifluoroacetic acid are added to a solution of 3.38 g (10.0 mmol) of ethyl [4-(2-tert-butoxy-2-oxoethoxy)-3-ethoxyphenyl]acetate in 25 ml of dichloromethane, and the mixture is stirred at RT until the reaction has gone to completion. Under reduced pressure, the solvent is removed from the reaction mixture, and the residue is then taken up in 100 ml of ethyl acetate and washed twice with 50 ml of water. The organic phase is dried over magnesium sulphate and, under reduced pressure, freed from the solvent. Purification on silica gel (mobile phase: cyclohexane/ethyl acetate 1:1) gives 2.54 g (89% of theory) of the desired product.

HPLC (Method 3): Rₜ=3.89 min.

¹H-NMR (200 MHz, CDCl₃): δ=1.25 (t, 3H), 1.48 (t, 3H), 3.55 (s, 2H), 4.15 (m, 4H), 4.65 (s, 2H), 6.88 (m, 3H), 7.65 (br. s, 1H).

### Example 7A

### [4-(2-tert-Butoxy-2-oxoethoxy)-3-ethoxyphenyl]acetic acid

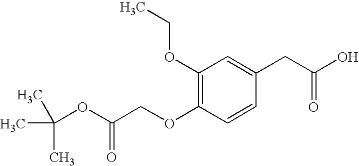

At RT, 11 ml (11.0 mmol) of 1M aqueous sodium hydroxide solution are added to a solution of 3.384 g (10.0 mmol) of ethyl [4-(2-tert-butoxy-2-oxoethoxy)-3-ethoxyphenyl acetate in 50 ml of ethanol. Until the reaction has gone to completion, the mixture is then stirred at RT. An equivalent amount of hydrochloric acid is added, the ethanol is distilled off under reduced pressure and the aqueous residue is extracted with ethyl acetate. The organic phase is dried over sodium sulphate and concentrated. Purification is carried out on silica gel (mobile phase: cyclohexane/ethyl acetate 5:1). This gives 451 mg (96% of theory) of the desired product.

HPLC (Method 4): Rₜ=3.06 min.

MS (DCI): m/z=328 [M+NH₄]⁺

¹H-NMR (200 MHz, CDCl₃): δ=1.4 (t, 3H), 1.5 (s, 9H), 3.55 (s, 2H), 4.1 (q, 2H), 4.55 (s, 2H), 6.78 (s, 2H), 6.85 (s, 1H).

### Example 8A

Ethyl [3-(2-tert-butoxy-2-oxoethoxy)phenyl]acetate

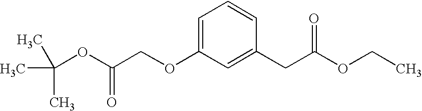

3.040 g (22.0 mmol) of potassium carbonate are added to a solution of 3.604 g (20.0 mmol) of ethyl (3-hydroxyphenyl)acetate in 25 ml of DMF, and the mixture is stirred at 50° C. for 1 h. 3.248 ml (22.0 mmol) of tert-butyl bromoacetate are then added, and stirring of the mixture at 50° C. is continued overnight. After the reaction has ended, the solvent is distilled off under reduced pressure. The crude product is taken up in 100 ml of ethyl acetate and washed three times with in each case 50 ml of water. The organic phase is dried over sodium sulphate, and the solvent is removed under reduced pressure. This gives 5.6 g (95% of theory) of the desired product.

HPLC (Method 3): Rₜ=4.75 min.

MS (DCI): m/z=312 [M+NH₄]⁺

¹H-NMR (200 MHz, CDCl₃): δ=1.25 (t, 3H), 1.5 (s, 9H), 3.55 (s, 2H), 4.15 (q, 2H), 4.5 (s, 2H), 6.82 (m, 3H), 7.25 (m, 1H).

### Example 9A

### [3-(2-Ethoxy-2-oxoethyl)phenoxy]acetic acid

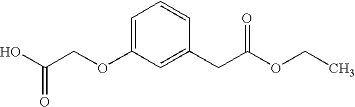

2.7 g (9.173 mmol) of ethyl [3-(2-tert-butoxy-2-oxoethoxy)phenyl]acetate are dissolved in 25 ml of dichloromethane, 7.07 ml (91.73 mmol) of trifluoroacetic acid are added at RT and the mixture is stirred at RT until the reaction has gone to completion. The reaction mixture is then concentrated under reduced pressure and the crude product is taken up in 100 ml of ethyl acetate and washed twice with in each case 50 ml of water. The organic phase is dried over magnesium sulphate and the solvent is removed under reduced pressure. Purification of the crude product on silica gel (mobile phase: cyclohexane/ethyl acetate 1:1) gives 2 g (91% of theory) of the desired product.

HPLC (Method 3): Rₜ=3.89 min.

MS (DCI): m/z=256 [M+NH₄]⁺

¹H-NMR (300 MHz, CDCl₃): δ=1.25 (t, 3H), 3.58 (s, 2H), 4.15 (q, 2H), 4.68 (s, 2H), 6.88 (m, 3H), 7.25 (t, 2H).

### Example 10A

### Ethyl [4-(2-tert-butoxy-2-oxoethoxy)-3-fluorophenyl]acetate

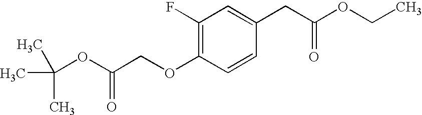

At RT, 844 mg (6.11 mmol) of potassium carbonate are added to a solution of 1.1 g (5.55 mmol) of ethyl (3-fluoro-4-hydroxyphenyl)acetate in 10 ml of DMF, and the suspension is stirred at 50° C. for 1 h. 0.90 ml (6.11 mmol) of tert-butyl bromoacetate is then added, and stirring of the mixture at 50° C. is continued overnight. After the reaction has ended, the solvent is distilled off under reduced pressure and the crude product is taken up in 100 ml of ethyl acetate and washed three times with in each case 50 ml of water. The organic phase is dried over sodium sulphate and the solvent is removed under reduced pressure. This gives 1.7 g (98% of theory) of the desired product.

HPLC (Method 3): Rₜ=4.93 min.

MS (DCI): m/z=330 [M+NH₄]⁺

¹H-NMR (200 MHz, CDCl₃): δ=1.25 (t, 3H), 1.48 (s, 9H), 3.5 (s, 2H), 4.15 (q, 2H), 4.58 (s, 2H), 6.95 (m, 3H).

### Example 11A

### [4-(2-Ethoxy-2-oxoethyl)-2-fluorophenoxy]acetic acid

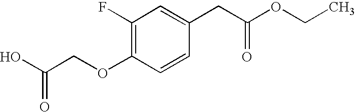

1.97 ml (2.15 mmol) of trifluoroacetic acid are added to a solution of 800 mg (0.215 mmol) of ethyl [4-(2-tert-butoxy-2-oxoethoxy)-3-fluorophenyl]acetate in 5 ml of dichloromethane, and the mixture is stirred at RT until the reaction has come to completion. The reaction mixture is then concentrated under reduced pressure and the residue is taken up in 50 ml of ethyl acetate and washed twice with in each case 20 ml of water. The organic phase is dried over magnesium sulphate and concentrated under reduced pressure, and the crude product is purified on silica gel (mobile phase: cyclohexane/ethyl acetate 1:1). This gives 590 mg (89% of theory) of the desired product.

HPLC (Method 2): Rₜ=5.17 min.

MS (DCI): m/z 501 [M+NH₄]⁺

¹H-NMR (200 MHz, DMSO-d₆): δ=−1.35 (s, 6H), 2.0 (s, 2H), 3.85 (s, 2H), 4.65 (s, 2H), 6.85 (m, 3H), 7.25 (t, 1H), 7.6 (m, 2H), 7.8 (dd, 4H), 8.15 (d, 1H), 13.0 (br. s, 1H).

### Example 12A

### 2-Chloro-4-(2-{3,3-dimethyl-5-[4-(trifluoromethyl)phenyl]-2,3-dihydro-1H-indol-1-yl}-2-oxoethyl)phenol

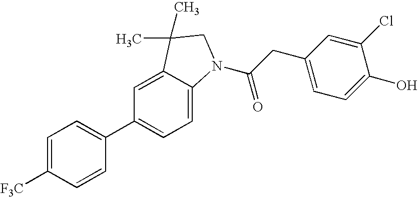

At 0° C., 287.6 mg (1.5 mmol) of N′-(3-dimethylaminopropyl)-N-ethylcarbodiimide hydrochloride are added to a solution of 218.5 mg (0.75 mmol) of 3,3-dimethyl-5-(4-trifluoromethyl)phenylindoline and 209.9 mg (1.13 mmol) of 3-chloro-4-hydroxyphenylacetic acid in 2 ml of 1,2-dichloroethane, and the mixture is then stirred at RT overnight. The reaction mixture is initially filtered through silica gel (mobile phase: cyclohexane/ethyl acetate 5:1). The product fraction is concentrated and then subjected to fine purification by preparative HPLC (RP18 column; mobile phase: acetonitrile/water, gradient 30:70-90:10). This gives 195 mg (56% of theory) of the desired product. HPLC (Method 5): Rₜ=3.39 min.

¹H-NMR (300 MHz, CDCl₃): δ=1.4 (s, 6H), 2.05 (s, 2H), 3.62 (br. s, 2H), 3.85 (br. s, 2H), 5.5 (s, 1H), 7.15 (d, 1H), 7.35 (d, 1H), 7.45 (d, 1H), 7.68 (s, 4H), 8.3 (d, 1H).

### Example 13A

### 4-(2-{3,3-Dimethyl-5-[4-(trifluoromethyl)phenyl]-2,3-dihydro-1H-indol-1-yl}-2-oxoethyl)-2-methoxyphenol

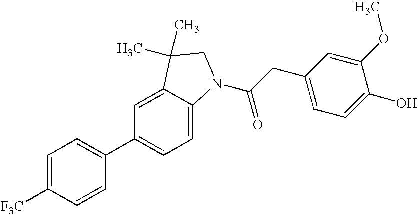

At 0° C., 287.6 mg (1.5 mmol) of N′-(3-dimethylaminopropyl)-N-ethylcarbodiimide hydrochloride are added to a solution of 218.5 mg (0.75 mmol) of 3,3-dimethyl-5-(4-trifluoromethyl)phenylindoline and 204.9 mg (1.3 mmol) of 4-hydroxy-3-methoxyphenylacetic acid in 2 ml of 1,2-dichloroethane, and the mixture is then stirred at RT overnight. The reaction mixture is directly purified on silica gel (mobile phase: cyclohexane/ethyl acetate 5:1). This gives 280 mg (82% of theory) of the desired product.

HPLC (Method 2): Rₜ=5.28 min.

MS (DCI): m/z=473 [M+NH₄]⁺

¹H-NMR (400 MHz, CDCl₃): δ=1.35 (s, 6H), 3.75 (s, 2H), 3.85 (s, 2H); 3.9 (s, 3H), 5.55 (s, 1H), 6.75 (d, 1H), 6.85 (m, 2H), 7.32 (s, 1H), 7.45 (d, 1H), 7.68 (s, 4H), 8.3 (d, 1H).

### WORKING EXAMPLES

### Example 1

### Ethyl [4-(2-{3,3-dimethyl-5-[4-(trifluoromethyl)phenyl]-2,3-dihydro-1H-indol-1-yl}-2-oxoethoxy)-3-ethoxyphenyl]acetate

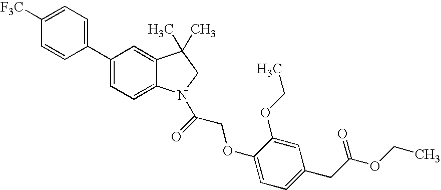

At 0° C., 131.6 mg (0.687 mmol) of N′-(3-dimethylaminopropyl)-N-ethylcarbodiimide hydrochloride are added to a solution of 100 mg (0.343 mmol) of 3,3-dimethyl-5-(4-trifluoromethyl)phenylindoline and 145.4 mg (0.515 mmol) of [2-ethoxy-4-(2-ethoxy-2-oxoethyl)phenoxy]acetic acid in 2 ml of 1,2-dichloroethane, and the reaction mixture is then stirred at RT overnight. The reaction mixture is purified directly on silica gel (mobile phase: cyclohexane/ethyl acetate 3:1). This gives 161 mg (84% of theory) of the desired product.

HPLC (Method 2): Rₜ=5.68 min.

MS (ESIpos): m/z=556 [M+H]⁺

¹H-NMR (300 MHz, CDCl₃): δ=1.45 (s, 12H), 3.5 (s, 2H), 4.05 (s, 2H), 4.15 (m, 4H), 4.8 (s, 2H), 6.78 (d, 1H), 6.85 (s, 1H), 6.95 (d, 1H), 7.35 (s, 1H), 7.45 (d, 1H), 7.68 (s, 4H), 8.28 (br. d, 1H).

### Example 2

### tert-Butyl [4-(2-{3,3-dimethyl-5-[4-(trifluoromethyl)phenyl]-2,3-dihydro-1H-indol-1-yl}-2-oxoethyl)-2-ethoxyphenoxy]acetate

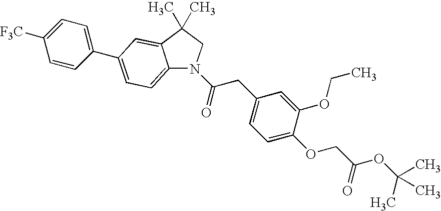

At 0° C., 159.9 mg (1.03 mmol) of N′-(3-dimethylaminopropyl)-N-ethylcarbodiimide hydrochloride are added to a solution of 150 mg (0.515 mmol) of 3,3-dimethyl-5-(4-trifluoromethyl)phenylindoline and 239.7 mg (0.772 mmol) of [4-(2-tert-butoxy-2-oxoethoxy)-3-ethoxyphenyl]acetic acid in 2 ml of 1,2-dichloroethane, and the mixture is then stirred at RT overnight. The reaction mixture is purified directly on silica gel (mobile phase: cyclohexane/ethyl acetate 5:1). This gives 279 mg (93% of theory) of the desired product.

HPLC (Method 6): Rₜ=4.82 min.

MS (ESIpos): m/z=584 [M+H]⁺

¹H-NMR (200 MHz, CDCl₃): δ=1.35 (s, 6H), 1.4 (t, 3H), 1.45 (s, 9H), 3.72 (s, 2H), 3.85 (s, 2H), 4.1 (q, 2H), 4.55 (s, 2H), 6.8 (s, 2H), 6.9 (s, 1H), 7.32 (s, 1H), 7.45 (d, 1H), 7.68 (s, 4H), 8.32 (d, 1H).

### Example 3

### Ethyl [4-(2-{3,3-dimethyl-5-[4-(trifluoromethyl)phenyl]-2,3-dihydro-1H-indol-1-yl}-2-oxoethoxy)-3-fluorophenyl]acetate

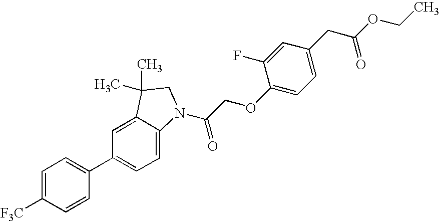

At 0° C., 131.6 mg (0.687 mmol) of N′-(3-dimethylaminopropyl)-N-ethylcarbodiimide hydrochloride are added to a solution of 100 mg (0.343 mmol) of 3,3-dimethyl-5-(4-trifluoromethyl)phenylindoline and 131.9 mg (0.515 mmol) of [4-(2-ethoxy-2-oxoethyl)-2-fluorophenoxy]acetic acid in 2 ml of 1,2-dichloroethane, and the mixture is then stirred at RT overnight. The mixture is directly purified on silica gel (mobile phase: cyclohexane/ethyl acetate 3:1). This gives 166 mg (91% of theory) of the desired product.

HPLC (Method 2): Rₜ=5.64 min.

MS (ESIpos): m/z 530 [M+H]⁺

¹H-NMR (300 MHz, CDCl₃): δ 1.25 (t, 3H), 1.42 (s, 6H), 3.52 (s, 2H), 3.98 (s, 2H), 4.15 (q, 2H), 4.82 (s, 2H), 7.05 (m, 3H), 7.35 (s, 1H), 7.45 (d, 1H), 7.68 (s, 4H), 8.28 (d, 1H).

### Example 4

### Ethyl [3-(2-{3,3-dimethyl-5-[4-(trifluoromethyl)phenyl]-2,3-dihydro-1H-indol-1-yl}-2-oxoethoxy)phenyl]acetate

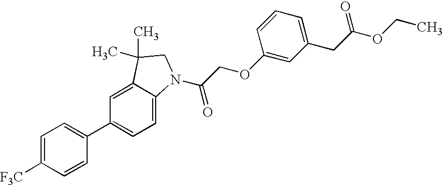

At 0° C., 131.6 mg (0.687 mmol) of N′-(3-dimethylaminopropyl)-N-ethylcarbodiimide hydrochloride are added to a solution of 100 mg (0.343 mmol) of 3,3-dimethyl-5-(4-trifluoromethyl)phenylindoline and 122.7 mg (0.515 mmol) of [3-(2-ethoxy-2-oxoethyl)phenoxy]acetic acid in 2 ml of 1,2-dichloroethane, and the mixture is then stirred at RT overnight. The reaction mixture is directly purified on silica gel (mobile phase: cyclohexane/ethyl acetate 3:1). This gives 171 mg (97% of theory) of the desired product.

HPLC (Method 2): Rₜ=5.63 min.

MS (ESIpos): m/z=512 [M+H]⁺

¹H-NMR (300 MHz, CDCl₃): δ=1.35 (t, 3H), 1.42 (s, 6H), 3.55 (s, 2H), 3.95 (s, 2H), 4.15 (q, 2H), 4.78 (s, 2H), 6.92 (m, 4H), 7.35 (s, 1H), 7.48 (d, 1H), 7.68 (s, 4H), 8.28 (d, 1H).

### Example 5

### [4-(2-{3,3-Dimethyl-5-[4-(trifluoromethyl)phenyl]-2,3-dihydro-1H-indol-1-yl}-2-oxoethyl)-2-ethoxyphenoxy]acetic acid

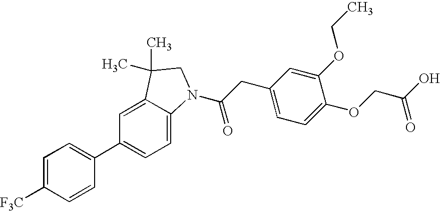

At RT, 0.154 ml (2.005 mmol) of trifluoroacetic acid is added to a solution of 117 mg (0.20 mmol) of tert-butyl [4-(2-{3,3-dimethyl-5-[4-(trifluoromethyl)phenyl]-2,3-dihydro-1H-indol-1-yl}-2-oxoethyl)-2-ethoxyphenoxy]acetate in 3 ml of dichloromethane, and the mixture is stirred until the reaction has gone to completion. The reaction mixture is then concentrated under reduced pressure and the crude product is purified on silica gel (mobile phase: cyclohexane/ethyl acetate 3:1, 1:1). This gives 75 mg (70% of theory) of the desired product.

HPLC (Method 3): Rₜ=5.20 min.

MS (ESI): m/z=528 [M+H]⁺

¹H-NMR (300 MHz, DMSO-d₆): δ=1.32 (t, 3H), 1.35 (s, 6H), 3.8 (s, 2H), 3.98 (s, 2H), 4.05 (q, 2H), 4.65 (s, 2H), 6.78 (m, 2H), 6.92 (s, 1H), 7.55 (d, 1H), 7.65 (s, 1H), 7.85 (dd, 4H), 8.12 (d, 1H), 12.88 (br. s, 1H).

### Example 6

### [4-(2-{3,3-Dimethyl-5-[4-(trifluoromethyl)phenyl]-2,3-dihydro-1H-indol-1-yl}ethyl)-2-methoxyphenoxy]acetic acid

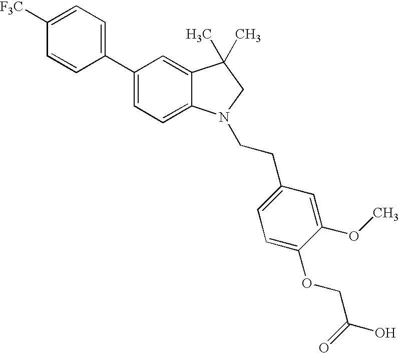

At RT, 0.022 ml (0.288 mmol) of trifluoroacetic acid is added to a solution of 16 mg (0.029 mmol) of tert-butyl [4-(2-{3,3-dimethyl-5-[4-(trifluoromethyl)phenyl]-2,3-dihydro-1H-indol-1-yl}ethyl)-2-methoxyphenoxy]acetate in 0.5 ml of dichloromethane, and the mixture is then stirred until the reaction has gone to completion. The reaction mixture is concentrated under reduced pressure and the crude product is purified on silica gel (mobile phase: dichloromethane→dichloromethane/methanol 10:1). This gives 13 mg (90% of theory) of the desired product.

MS (ESI): m/z=500 [M+H]⁺

¹H-NMR (300 MHz, CDCl₃): δ=1.32 (s, 6H), 2.85 (t, 2H), 3.2 (s, 2H), 3.38 (t, 2H), 3.88 (s, 3H), 4.6 (s, 2H), 6.48 (d, 1H), 6.8 (s, 2H), 6.9 (d, 1H), 7.22 (s, 1H), 7.35 (d, 1H), 7.6 (s, 4H).

### Example 7

### tert-Butyl [2-chloro-4-(2-{3,3-dimethyl-5-[4-(trifluoromethyl)phenyl]-2,3-dihydro-1H-indol-1-yl}-2-oxoethyl)phenoxy]acetate

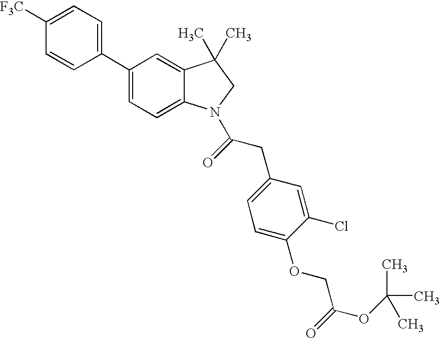

30 mg (0.217 mmol) of potassium carbonate are added to a solution of 100 mg (0.217 mmol) of 2-chloro-4-(2-{3,3-dimethyl-5-[4-(trifluoromethyl)phenyl]-2,3-dihydro-1H-indol-1-yl}-2-oxoethyl)phenol in 2 ml of DMF, and the mixture is stirred at RT for 1 h. 0.035 ml (0.239 mmol) of tert-butyl bromoacetate is then added. The mixture is stirred initially at RT for 3 h and then at 50° C. overnight. After the reaction has ended, the solvent is distilled off under reduced pressure and the crude product is taken up in 30 ml of ethyl acetate and washed twice with in each case 15 ml of water and once with saturated potassium carbonate solution. The organic phase is dried over sodium sulphate and concentrated under reduced pressure. This gives 117 mg (93% of theory) of the desired product.

MS (ESI): m/z=575 [M+H]⁺

¹H-NMR (300 MHz, CDCl₃): δ=1.38 (s, 6H), 1.5 (s, 9H), 3.75 (s, 2H), 3.85 (s, 2H), 4.58 (s, 2H), 6.82 (d, 1H), 7.15 (d, 1H), 7.35 (d, 2H), 7.48 (d, 1H), 7.68 (s, 4H), 8.3 (d, 1H).

### Example 8

### tert-Butyl [4-(2-{3,3-dimethyl-5-[4-(trifluoromethyl)phenyl]-2,3-dihydro-1H-indol-1-yl}-2-oxoethyl)-2-methoxyphenoxy]acetate

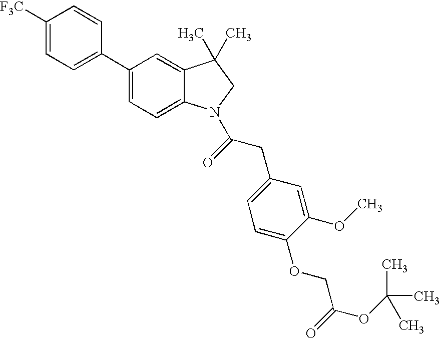

30.343 mg (0.22 mmol) of potassium carbonate are added to a solution of 100 mg (0.22 mmol) of 4-(2-{3,3-dimethyl-5-[4-(trifluoromethyl)phenyl]-2,3-dihydro-1H-indol-1-yl}-2-oxoethyl)-2-methoxyphenol in 2 ml of DMF, and the mixture is stirred at RT for 1 h. 0.036 ml (0.242-mmol) of tert-butyl bromoacetate are then added. The mixture is then initially stirred at RT for 3 h and then further at 50° C. overnight. After the reaction has ended, the solvent is distilled off under reduced pressure and the crude product is taken up in 30 ml of ethyl acetate and washed twice with in each case 15 ml of water and once with saturated potassium carbonate solution. The organic phase is dried over sodium sulphate and concentrated under reduced pressure. Purification of the crude product on silica gel (mobile phase: cyclohexane/ethyl acetate 3:1) gives 111 mg (88% of theory) of the desired product.

HPLC (Method 2): Rₜ=5.62 min.

MS (ESI): m/z=514 [M+H]⁺P ¹H-NMR (200 MHz, CDCl₃): δ=1.35 (s, 6H), 1.45 (s, 9H), 3.75 (s, 2H), 3.88 (s, 2H), 3.9 (s, 3H), 4.55 (s, 2H), 6.78 (s, 2H), 6.9 (s, 1H), 7.35 (s, 1H), 7.45 (d, 1H), 7.68 (s, 4H), 8.3 (d, 1H).

### Example 9

### [4-(2-{3,3-Dimethyl-5-[4-(trifluoromethyl)phenyl]-2,3-dihydro-1H-indol-1-yl}-2-oxoethoxy)-3-ethoxyphenyl]acetic acid

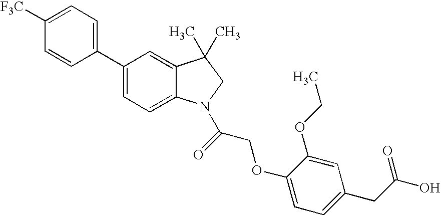

At RT, 0.257 ml (0.257 mmol) of 1 M aqueous sodium hydroxide solution is added to a solution of 130 mg (0.234 mmol) of ethyl [4-(2-{3,3-dimethyl-5-[4-(trifluoromethyl)phenyl]-2,3-dihydro-1H-indol-1-yl}-2-oxoethoxy)-3-ethoxyphenyl]acetate in 5 ml of ethanol, and the mixture is then stirred at RT until the reaction has gone to completion. 0.3 ml of 1 N hydrochloric acid is then added, and the ethanol is distilled off under reduced pressure. A white precipitate is obtained, which is filtered off with suction, washed with water and dried under reduced pressure. This gives 117 mg (94% of theory) of the desired product.

HPLC (Method 3): Rₜ=5.2 min.

MS (ESI): m/z=528 [M+H]⁺

¹H-NMR (200 MHz, DMSO-d₆): δ=1.35 (t, 3H), 1.4 (s, 6H), 3.5 (s, 2H), 4.0 (s, 2H), 4.05 (q, 2H), 4.95 (s, 2H), 6.75 (d, 1H), 6.9 (s, 2H), 7.6 (d, 1H), 7.7 (s, 1H), 7.85 (dd, 4H), 8.12 (d, 1H), 12.25 (br. s, 1H);

### Example 10

### [3-(2-{3,3-Dimethyl-5-[4-(trifluoromethyl)phenyl]-2,3-dihydro-1H-indol-1-yl}-2-oxoethoxy)phenyl]acetic acid

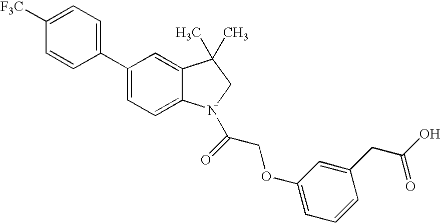

At RT, 0.187 ml (0.187 mmol) of 1 M aqueous sodium hydroxide solution are added to a solution of 87 mg (0.17 mmol) of ethyl [3-(2-{3,3-dimethyl-5-[4-(trifluoromethyl)phenyl]-2,3-dihydro-1H-indol-1-yl}-2-oxoethoxy)phenyl]acetate in 5 ml of ethanol, and the mixture is stirred at RT until the reaction has gone to completion. 0.25 ml of 1 N hydrochloric acid is then added, and the ethanol is distilled off under reduced pressure. A white precipitate is obtained, which is filtered off with suction, washed with water and dried under reduced pressure. This gives 75 mg (91% of theory) of the desired product.

HPLC (Method 3): Rₜ=5.16 min.

MS (ESI): m/z=484 [M+H]⁺

¹H-NMR (200 MHz, DMSO-d₆): δ=1.4 (s, 6H), 3.55 (s, 2H), 4.0 (s, 2H), 4.95 (s, 2H), 6.88 (d, 1H), 6.92 (s, 2H), 7.25 (t, 1H), 7.6 (d, 1H), 7.72 (s, 1H), 7.85 (dd, 4H), 8.12 (d, 1H), 12.35 (br. s, 1H).

### Example 11

### Ethyl [4-(2-{3,3-dimethyl-5-[4-(trifluoromethyl)phenyl]-2,3-dihydro-1H-indol-1-yl}ethoxy)-3-fluorophenyl]acetate

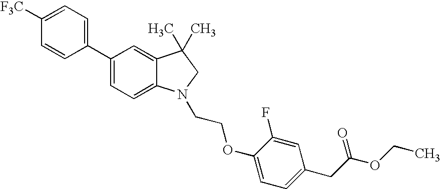

At RT, 0.06 ml (0.326 mmol) of diphenylsilane is added to a solution of 69 mg (0.13 mmol) of ethyl [4-(2-{3,3-dimethyl-5-[4-(trifluoromethyl)phenyl]-2,3-dihydro-1H-indol-1-yl}-2-oxoethoxy)-3-fluorophenyl]acetate and 1.2 mg (0.001 mmol) of carbonyl-tris(triphenylphosphine)rhodium(I) hydride in 1 ml of THF, and the mixture is stirred at RT overnight. The mixture is concentrated under reduced pressure and purified on silica gel (mobile phase: cyclohexane/ethyl acetate 7:1). The product is used directly for the next step.

### Example 12

### [4-(2-{3,3-Dimethyl-5-[4-(trifluoromethyl)phenyl]-2,3-dihydro-1H-indol-1-yl}ethoxy)-3-fluorophenyl]acetic acid hydrochloride

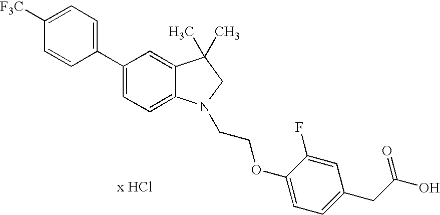

1.43 ml (0.143 mmol) of 0.1 M aqueous sodium hydroxide solution are added to a solution of 67 mg (0.13 mmol) of ethyl [4-(2-{3,3-dimethyl-5-[4-(trifluoromethyl)phenyl]-2,3-dihydro-1H-indol-1-yl}ethoxy)-3-fluorophenyl]acetate in 2 ml of ethanol, and the mixture is stirred at RT until the reaction has gone to completion. An equivalent amount of hydrochloric acid is then added, the aqueous phase is extracted with ethyl acetate and the organic phase is dried over sodium sulphate and concentrated under reduced pressure. The crude product is purified on silica gel 60 (mobile phase: cyclohexane/ethyl acetate 5:1). The product fractions are concentrated under reduced pressure and taken up in ethyl acetate, and a solution of hydrogen chloride in dioxane is added. The mixture is, under reduced pressure, evaporated to dryness. This gives 31 mg (45% of theory) of the desired product.

HPLC (Method 3): Rₜ 5.66 min.

MS (ESI): m/z 488 [M+H]⁺

¹H-NMR (200 MHz, DMSO-d₆): δ=1.28 (s, 6H), 3.35 (s, 2H), 3.49 (s, 2H), 3.95 (br. s, 3H), 4.25 (t, 2H), 6.65 (d, 1H), 7.1 (m, 3H), 7.42 (m, 2H), 7.75 (dd, 4H).

### Example 13

### Ethyl [3-(2-{3,3-dimethyl-5-[4-(trifluoromethyl)phenyl]-2,3-dihydro-1H-indol-1-yl}ethoxy)phenyl]acetate

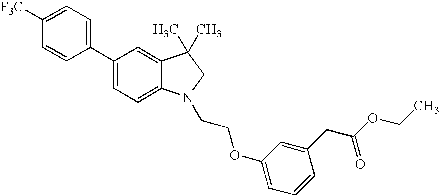

At RT, 0.068 ml (0.367 mmol) of diphenylsilane are added to a solution of 75 mg (0.147 mmol) of ethyl [3-(2-{3,3-dimethyl-5-[4-(trifluoromethyl)phenyl]-2,3-dihydro-1H-indol-1-yl}-2-oxoethoxy)phenyl]acetate and 1.3 mg (0.001 mmol) of carbonyl-tris(triphenylphosphine)rhodium(I) hydride in 1 ml of THF, and the mixture is stirred at RT overnight. The mixture is then concentrated under reduced pressure and purified on silica gel (mobile phase: cyclohexane/ethyl acetate 7:1). The product is used directly for the next step.

### Example 14

### [3-(2-{3,3-Dimethyl-5-[4-(trifluoromethyl)phenyl]-2,3-dihydro-1H-indol-1-yl}ethoxy)phenyl]acetic acid hydrochloride

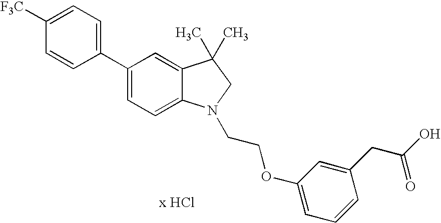

At RT, 1.62 ml (0.162 mmol) of 0.1 M aqueous sodium hydroxide solution are added to a solution of 73.14 mg (0.147 mmol) of ethyl [3-(2-{3,3-dimethyl-5-[4-(trifluoromethyl)phenyl]-2,3-dihydro-1H-indol-1-yl}ethoxy)phenyl]acetate in 2 ml of ethanol, and the mixture is then stirred at RT until the reaction has gone to completion. An equivalent amount of hydrochloric acid is then added, the aqueous phase is extracted with ethyl acetate and the organic phase is dried over sodium sulphate and concentrated under reduced pressure. The crude product is purified on silica gel 60 (mobile phase: cyclohexane/ethyl acetate 5:1). The product fractions are concentrated, the residue is taken up in ethyl acetate, a solution of hydrogen chloride in dioxane is added and the mixture is once more evaporated to dryness, giving 30 mg (40% of theory) of the desired product.

HPLC (Method 3): Rₜ 5.67 min.

MS (ESI): m/z=470 [M+H]⁺

¹H-NMR (200 MHz, DMSO-d₆): δ=1.3 (s, 6H), 3.52 (s, 2H), 3.55 (s, 2H), 3.58 (t, 2H), 4.18 (t, 2H), 6.68 (d, 1H), 6.85 (m, 3H), 7.25 (t, 1H), 7.42 (m, 2H), 7.75 (dd, 4H), 12.3 (br. s, 1H).

### Example 15

### tert-Butyl [4-(2-{3,3-dimethyl-5-[4-(trifluoromethyl)phenyl]-2,3-dihydro-1H-indol-1-yl}ethyl)-2-methoxyphenoxy]acetate

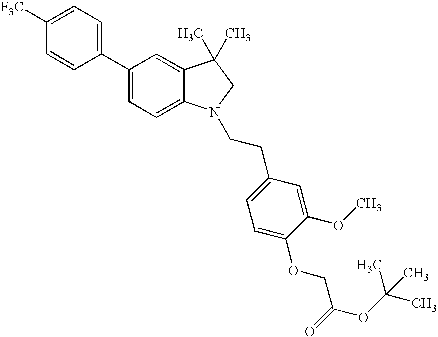

At RT, 0.061 ml (0.329 mmol) of diphenylsilane are added to a solution of 75 mg (0.132 mmol) of tert-butyl [4-(2-{3,3-dimethyl-5-[4-(trifluoromethyl)phenyl]-2,3-dihydro-1H-indol-1-yl}-2-oxoethoxy)-2-methoxyphenoxy]acetate and 1.2 mg (0.001 mmol) of carbonyl-tris(triphenylphosphine)rhodium(I) hydride in 1 ml of THF, and the mixture is stirred at RT overnight. The mixture is then concentrated under reduced pressure, pre-purified on silica gel (mobile phase: cyclohexane/ethyl acetate 7:1) and fine-purified by preparative HPLC (RP18 column; mobile phase: acetonitrile/water, gradient 30:70→90:10). This gives 20 mg (27% of theory) of the desired product.

MS (ESI): m/z=555 [M+H]⁺

¹H-NMR (300 MHz, CDCl₃): δ 1.35 (s, 6H), 1.45 (s, 9H), 2.88 (t, 2H), 3.22 (s, 2H), 3.4 (t, 2H), 3.88(s, 3H), 4.55 (s, 2H), 6.52 (d, 1H), 6.75 (s, 2H), 6.8 (s, 1H), 7.22 (s, 1H), 7.35 (d, 1H), 7.62 (s, 4H).

### Example 16

### tert-Butyl [2-chloro-4-(2-{3,3-dimethyl-5-[4-(trifluoromethyl)phenyl]-2,3-dihydro-1H-indol-1-yl}ethyl)phenoxy]acetate

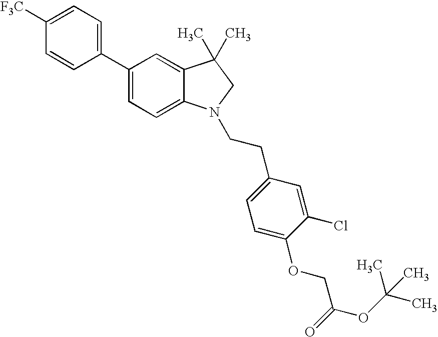

At RT, 0.068 ml (0.366 mmol) of diphenylsilane is added to a solution of 84 mg (0.146 mmol) of tert-butyl [2-chloro-4-(2-{3,3-dimethyl-5-[4-(trifluoromethyl)phenyl]-2,3-dihydro-1H-indol-1-yl}-2-oxoethyl)phenoxy]acetate and 1.3 mg (0.001 mmol) of carbonyl-tris(triphenylphosphine)rhodium(I) hydride in 1 ml of THF, and the mixture is stirred at RT overnight. The mixture is then concentrated under reduced pressure, pre-purified on silica gel (mobile phase: cyclohexane/ethyl acetate 7:1) and fine-purified twice by preparative HPLC (RP18 column; mobile phase: acetonitrile/water, gradient 30:70→90:10). This gives 29.5 mg (36% of theory) of the desired product.

MS (ESI): m/z=559 [M+H]⁺

¹H-NMR (300 MHz, CDCl₃): δ=1.35 (s, 6H), 1.48 (s, 9H), 2.88 (t, 2H), 3.25 (s, 2H), 3.38 (t, 2H), 4.55 (s, 2H), 6.6 (d, 1H), 6.78 (d, 1H), 7.08 (d, 1H), 7.3 (s, 1H), 7.38 (d, 1H), 7.6 (s, 4H), 7.65 (s, 1H).

### B. ASSESSMENT OF THE PHYSIOLOGICAL ACTIVITY

### Example A

Cellular Transactivation Assay:

Test Principle:

A cellular assay is used to identify activators of the peroxisome proliferator-activated receptor delta (PPAR-delta).

Since mammalian cells contain different endogenous nuclear receptors which may complicate an unambiguous interpretation of the results, an established chimera system is used in which the ligand binding domain of the human PPARδ receptor is fused to the DNA binding domain of the yeast transcription factor GAL4. The resulting GAL4-PPARδ chimera is co-transfected and stably expressed in CHO cells having a reporter construct.

Cloning:

The GAL4-PPARδ expression construct contains the ligand binding domain of PPARδ (amino acids 414-1326), which is PCR-amplified and cloned into the vector pcDNA3.1. This vector already contains the GAL4 DNA binding domain (amino acids 1-147) of the vector pFC2-dbd (Stratagene). The reporter construct, which contains five copies of the GAL4 binding site upstream of a thymidine kinase promoter, expresses firefly luciferase (Photinus pyralis) following activation and binding of GAL4-PPARδ.

Transactivation Assay (Luciferase Reporter):

CHO (chinese hamster ovary) cells are sown in CHO-A-SFM medium (GIBCO), supplemented by 2.5% foetal calf serum and 1% penicillin/streptomycin (GIBCO), at a cell density of 2×10³ cells per well in a 384-well plate (Greiner). The cells are cultivated at 37° C. for 48 h and then stimulated. To this end, the substances to be tested are taken up in the abovementioned medium and added to the cells. After a stimulation period of 24 hours, the luciferase activity is measured using a video camera. The relative light units measured give, as a function of the substance concentration, a sigmoidal stimulation curve. The EC₅₀ values are calculated using the computer program GraphPad PRISM (Version 3.02).

In this test, Working Examples 1-16 show EC₅₀ values in a range of from 10 nM to 10 μM.

### Example B

Descriptions of the Test for Finding Pharmacologically Active Substances Which Increase HDL Cholesterol (HDL-C) Concentrations in the Serum of Transgenic Mice Transfected with the Human ApoA1 Gene (hApoA1) and/or have an Effect on the Metabolic Syndrome of Adipose ob,ob Mice and Lower Their Blood Glucose Concentration:

The substances to be examined in vivo for their HDL-C-increasing activity are administered orally to male transgenic hApoA1 mice. One day prior to the start of the experiment, the animals are randomized into groups with the same number of animals, generally n=7-10. Throughout the experiment, the animals have drinking water and feed ad libitum. The substances are administered orally once a day for 7 days. To this end, the test substances are dissolved in a solution of Solutol HS 15+ethanol+saline (0.9%) in a ratio of 1+1+8 or in a solution of Solutol HS 15+saline (0.9%) in a ratio of 2+8. The dissolved substances are administered in a volume of 10 ml/kg of body weight using a stomach tube. Animals which have been treated in exactly the same manner but have only been given the solvent (10 ml/kg of body weight), without test substance, serve as control group.

Prior to the first administration of substance, a blood sample from each of the mice is taken by puncture of the retroorbital venous plexus, to determine ApoA1, serum cholesterol, HDL-C and serum triglycerides (TG) (zero value). Subsequently, using a stomach tube, the test substance is administered for the first time to the animals. 24 hours after the last administration of substance (i.e. on day 8 after the start of the treatment), another blood sample is taken from each animal by puncture of the retroorbital venous plexus, to determine the same parameters. The blood samples are centrifuged and, after the serum has been obtained, cholesterol and TG are determined photometrically using an EPOS Analyzer 5060 (Eppendorf-Gerätebau, Netheler & Hinz GmbH, Hamburg). The said determinations are carried out using commercial enzyme tests (Boehringer Mannheim, Mannheim).

To determine the HDL-C, the non-HDL-C fraction is precipitated using 20% PEG 8000 in 0.2 M glycine buffer pH 10. From the supernatant, the cholesterol is determined UV-photometrically (BIO-TEK Instruments, USA) in a 96-well plate using a commercial reagent (Ecoline 25, Merck, Darmstadt).

Human mouse-ApoA1 is determined with a Sandwich ELISA method using a polyclonal anti-human-ApoA1 antibody and a monoclonal anti-human-ApoA1 antibody (Biodesign International, USA). Quantification is carried out UV-photometrically (BIO-TEK Instruments, USA) using peroxidase-coupled anti-mouse-IGG antibodies (KPL, USA) and peroxidase substrate (KPL, USA).

The effect of the test substances on the HDL-C concentration is determined by subtracting the value measured for the 1st blood sample (zero value) from the value measured for the 2nd blood sample (after the treatment). The mean of the differences of all HDL-C values of one group is determined and compared to the mean of the differences of the control group.

Statistical evaluation is carried out using Student's t-test, after the variances have been checked for homogeneity.

Substances which increase the HDL-C of the treated animals in a statistically significant (p<0.05) manner by at least 15%, compared to that of the control group, are considered to be pharmacologically effective.

To examine substances for their effect on a metabolic syndrome, animals having an insulin resistance and increased blood glucose levels are used. To this end, C57Bl/6J Lep <ob> mice are treated using the same protocol as for the transgenic ApoA1 mice. The serum lipids are determined as described above. In these animals, serum glucose is additionally determined, as a parameter for blood glucose. Serum glucose is determined enzymatically in an EPOS Analyzer 5060 (see above), using commercially available enzyme tests (Boehringer Mannheim).

A blood-glucose-lowering effect of the test substances is determined by subtracting the value measured for the 1st blood sample of an animal (zero value) from the value measured for the 2nd blood sample of the same animal (after the treatment). The mean of the differences of all serum glucose values of one group is determined and compared to the mean of the differences of the control group.

Statistical evaluation is carried out using Student's t-test, after the variances have been checked for homogeneity.

Substances which lower the serum glucose concentration of the treated animals in a statistically significant (p<0.05) manner by at least 10%, compared to that of the control group, are considered to be pharmacologically effective.

### C. WORKING EXAMPLES OF PHARMACEUTICAL COMPOSITIONS

The compounds according to the invention can be converted into pharmaceutical preparations in the following ways:

Tablet:

Composition:

100 mg of the compound of Example 1, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

Production:

The mixture of the compound according to the invention, lactose and starch is granulated with a 5% strength solution (m/m) of the PVP in water. The granules are dried and then mixed with the magnesium stearate for 5 minutes. This mixture is compressed using a conventional tablet press (see above for format of the tablet). A compressive force of 15 kN is used as guideline for the compression.

Suspension Which can be Administered Orally:

Composition:

1000 mg of the compound of Example 1, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

Production:

The Rhodigel is suspended in ethanol, and the active compound is added to the suspension. The water is added while stirring. The mixture is stirred for about 6 h until the swelling of the Rhodigel is complete.

Solution Which can be Administered Orally:

Composition:

500 mg of the compound of Example 1, 2.5 g of polysorbate and 97 g of polyethylene glycol 400.20 g of oral solution correspond to a single dose of 100 mg of the compound according to the invention.

Production:

The compound according to the invention is suspended in the mixture of polyethylene glycol and polysorbate with stirring. Stirring is continued until the compound according to the invention has dissolved completely.

1. Compounds of the general formula (I) 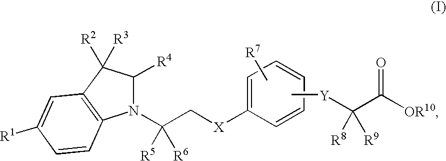
in which
R¹ represents phenyl or represents 5- or 6-membered heteroaryl having up to two heteroatoms from the group consisting of N, O and/or S, which radicals may for their part each be substituted by one to three identical or different substitutents selected from the group consisting of halogen, cyano, nitro, (C₁-C₆)-alkyl (which for its part may be substituted by hydroxyl), (C₁-C₆)-alkoxy, trifluoromethyl, trifluoromethoxy, (C₁-C₆)-alkylsulphonyl, (C₁-C₆)-alkanoyl, (C₁-C₆)-alkoxycarbonyl, carboxyl, amino, (C₁-C₆)-acylamino, mono- and di-(C₁-C₆)-alkylamino,
R² and R³ are identical or different and independently of one another represent hydrogen or (C₁-C₄)-alkyl or together with the carbon atom to which they are attached form a 3- to 7-membered spiro-linked cycloalkyl ring,
R⁴ represents hydrogen or (C₁-C₄)-alkyl,
R⁵ and R⁶ represent hydrogen or together with the carbon atom to which they are attached form a carbonyl group,
R⁷ represents hydrogen, (C₁-C₄)-alkyl, (C₁-C₄)-alkoxy or halogen,
R⁸ and R⁹ are identical or different and independently of one another represent hydrogen or (C₁-C₄)-alkyl,
R¹⁰ represents hydrogen or represents a hydrolyzable group which may be degraded to the corresponding carboxylic acid,
X represents O, S or N—R¹¹
and
Y represents a bond
or
X represents a bond
and
Y represents O, S or N—R¹, where
R¹¹ represents in each case hydrogen, (C₁-C₄)-alkyl or (C₁-C₄)-alkanoyl,
and their pharmaceutically acceptable salts, solvates and solvates of the salts.

2. Compounds of the general formula (I) according to claim 1 in which
R¹ represents phenyl which may be mono- or disubstituted by identical or different substitutents selected from the group consisting of halogen, cyano, nitro, (C₁-C₄)-alkyl (which for its part may be substituted by hydroxyl), (C₁-C₄)-alkoxy, trifluoromethyl, trifluoromethoxy, (C₁-C₄)-alkanoyl, amino, mono- and di-(C₁-C₄)-alkylamino,
R² and R³ are identical or different and represent (C₁-C₄)-alkyl or together with the carbon atom to which they are attached form a 4- to 6-membered spiro-linked cycloalkyl ring,
R⁴ represents hydrogen,
R⁵ and R⁶ represent hydrogen or together with the carbon atom to which they are attached form a carbonyl group,
R⁷ represents hydrogen, (C₁-C₄)-alkyl, (C₁-C₄)-alkoxy, fluorine or chlorine,
R⁸ and R⁹ independently of one another represent hydrogen or methyl,
R¹⁰ represents hydrogen,
X represents O or S
and
Y represents a bond
or
X represents a bond
and
Y represents O or S.

3. Compounds of the general formula (I) according to claim 1 in which
R¹ represents phenyl which may be substituted by fluorine, chlorine, cyano, methyl, ethyl, tert-butyl, methoxy, ethoxy, trifluoromethyl, trifluoromethoxy, amino, dimethylamino or diethylamino,
R² and R³ each represent methyl or together with the carbon atom to which they are attached form a spiro-linked cyclopentane or cyclohexane ring,
R⁴ represents hydrogen,
R⁵ and R⁶ represent hydrogen or together with the carbon atom to which they are attached form a carbonyl group,
R⁷ represents hydrogen, methyl, methoxy, ethoxy, fluorine or chlorine,
R⁸ and R⁹ each represent hydrogen,
R¹⁰ represents hydrogen,
X represents O
and
Y represents a bond
or
X represents a bond
and
Y represents O.

4. Compounds of the formula (I-A) 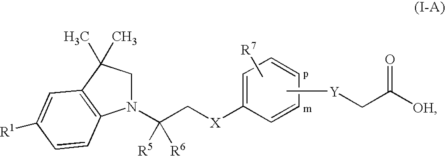
in which
R¹ represents phenyl which is substituted by fluorine, chlorine or trifluoromethyl,
R⁵ and R⁶ represent hydrogen or together with the carbon atom to which they are attached form a carbonyl group,
R⁷ represents hydrogen, methyl, methoxy, ethoxy, fluorine or chlorine,
X represents O
and
Y represents a bond
or
X represents a bond
and
Y represents O,
and the group attached via Y is located in the para- or meta-position (marked in formula (I-A)) of the phenyl ring relative to the substitutent X.

5. Process for preparing the compounds of the general formula (I) or (I-A) as defined in claims 1 to 4, characterized in that compounds of the formula (II) 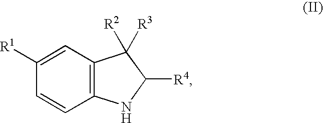
in which R¹, R², R³ and R⁴ are each as defined above
are either
[A] coupled in an inert solvent in the presence of a condensing agent and if appropriate in the presence of an auxiliary base with a compound of the formula (III) 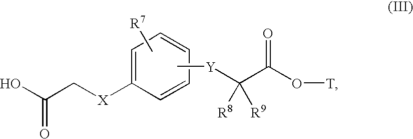
in which
X, Y, R⁷, R⁸ and R⁹ are each as defined above and
T represents benzyl or (C₁-C₆)-alkyl,
to give compounds of the formula (IV) 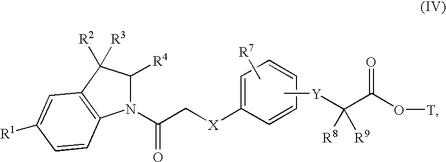
in which R¹, R², R³, R⁴, R⁷, R⁸, R⁹, X, Y and T are each as defined above,
which, if R⁵ and R⁶ in formula (I) or (I-A) represent hydrogen are, in an inert solvent in the presence of a suitable reducing agent, converted further into compounds of the formula (V) 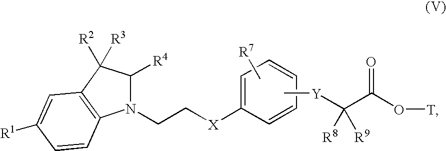
in which R¹, R², R³, R⁴, R⁷, R⁸, R⁹, X, Y and T are each as defined above,
the compounds of the formula (IV) or (V) are then converted with acids or bases or, if T represents benzyl, also hydrogenolytically, into the corresponding carboxylic acids of the formula (VI) 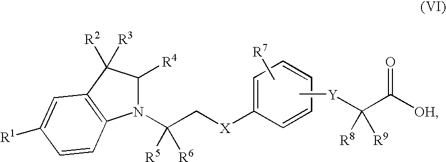
in which R¹, R², R³, R⁴, R⁵, R⁶, R⁷, R⁸, R⁹, X and Y are each as defined above,
or else
[B] in the case that in formula (I) or (I-A) X represents a bond and Y represents O, S or N—R¹¹, initially coupled in an inert solvent in the presence of a condensating agent and if appropriate in the presence of an auxiliary base with a compound of the formula (VII) 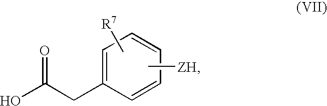
in which
R⁷ is as defined above and
Z represents O, S or N—R¹¹, where R¹¹ is as defined above,
to give compounds of the formula (VIII) 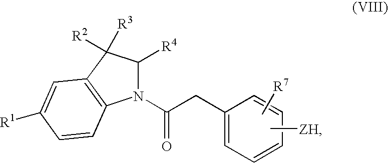
in which R¹, R², R³, R⁴, R⁷ and Z are each as defined above,
which are then, in an inert solvent in the presence of a base, reacted with a compound of the formula (IX) 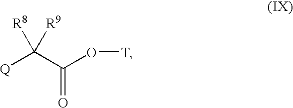
in which R⁸, R⁹ and T are each as defined above and
Q represents a suitable leaving group, such as, for example, halogen, mesylate or tosylate,
to give compounds of the formula (X) 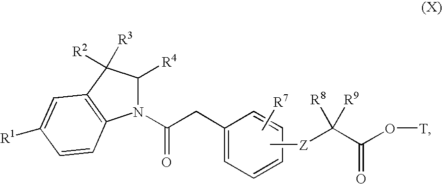
in which R¹, R², R³, R⁴, R⁷, R⁸, R⁹, T and Z are each as defined above,
which, if R⁵ and R⁶ in formula (I) or (I-A) represent hydrogen, are, in an inert solvent in the presence of a suitable reducing agent, converted further into compounds of the formula (XI) 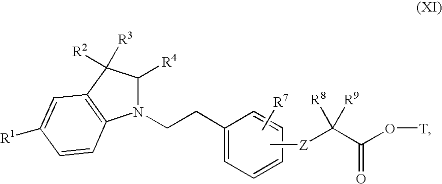
in which R¹, R², R³, R⁴, R⁷, R⁸, R⁹, T and Z are each as defined above,
the compounds of the formula (X) or (XI) are then, with acids or bases or, if T represents benzyl, also hydrogenolytically, converted into the corresponding carboxylic acids of the formula (XII) 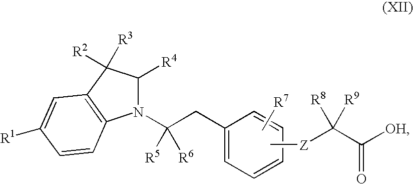
in which R¹, R², R³, R⁴, R⁵, R⁶, R⁷, R⁸, R⁹ and Z are each as defined above,
the carboxylic acids of the formula (VI) or (XII) are, if appropriate, modified further into compounds of the formula (I) or (I-A) using known esterification methods,
and the resulting compounds of the formula (VI), (XII), (I) or (I-A) are, if appropriate, converted into their solvates, salts and/or solvates of the salts using the corresponding (i) solvents and/or (ii) bases or acids.

6. Compounds of the formula (I) or (I-A), as defined in claims 1 to 4 for the prophylaxis and treatment of diseases.

7. Medicaments, comprising at least one compound of the formula (I) or (I-A) as defined in claims 1 to 4 and inert non-toxic pharmaceutically suitable carriers, auxiliaries, solvents, vehicles, emulsifiers and/or dispersants.

8. Use of compounds of the formula (I) or (I-A) and medicaments as defined in claims 1 to 7 for the prophylaxis and/or treatment of diseases.

9. Use of compounds of the formula (I) or (I-A) as defined in claims 1 to 6 for preparing medicaments.

10. Use of compounds of the formula (I) or (I-A) as defined in claims 1 to 4 for preparing medicaments for the prophylaxis and treatment of stroke, arteriosclerosis, coronary heart diseases and dyslipidaemia, for the prophylaxis of myocardial infarction and for the treatment of restenosis after coronary angioplasty or stenting.

11. Method for the prophylaxis and treatment of diseases, characterized in that compounds of the formula (I) or (I-A) as defined in claims 1 to 4 are allowed to act on living beings.
